(19) United States
(12) Patent Application Publication
(10) Pub. No.: US 2022/0023413 A1
(43) Pub. Date: Jan. 27, 2022

(54) RECOMBINANT MUMPS VIRUS VACCINE EXPRESSING GENOTYPE G FUSION AND HEMAGGLUTININ-NEURAMINIDASE PROTEINS

(71) Applicant: The U.S.A., as represented by the Secretary, Department of Health and Human Services, Silver Spring, MD (US)

(72) Inventors: Steven A. Rubin, Buckeystown, MD (US); Trent J. Bosma, Silver Spring, MD (US); Christian J. Sauder, Rockville, MD (US); Tahir H. Malik, Silver Spring, MD (US)

(73) Assignee: The U.S.A., as represented by the Secretary, Department of Health and Human Services, Silver Spring, MD (US)

(21) Appl. No.: 17/413,331

(22) PCT Filed: Dec. 12, 2019
(86) PCT No.: PCT/US2019/065926

§ 371 Date: Jun. 11, 2021

Related U.S. Application Data
(60) Provisional application No. 62/778,675, filed on Dec. 12, 2018.

(51) Int. Cl. A61K 39/165; C12N 15/86; C12N 7/00; A61P 31/14

(52) U.S. Cl. CPC A61K 39/165; C12N 15/86; C12N 7/00; A61K 2039/522; C12N 2760/18721; C12N 2760/18734; A61P 31/14

(57) ABSTRACT

A recombinant, attenuated mumps virus is described. The recombinant virus is based on the genotype A Jeryl Lynn vaccine strain, but is modified to express genotype G consensus fusion (F) and hemagglutinin-neuraminidase (HN) proteins. The recombinant virus optionally includes a mutation that prevents expression of viral protein V. The recombinant mumps virus can be used as a vaccine to inhibit mumps virus infection and the development of mumps disease.

### CROSS REFERENCE TO RELATED APPLICATIONS

This application claims the benefit of U.S. Provisional Application No. 62/778,675, filed Dec. 12, 2018, which is herein incorporated by reference in its entirety.

### ACKNOWLEDGMENT OF GOVERNMENT SUPPORT

This invention was made with government support under project number Z01 BK 08015-12 awarded by the National Institutes of Health. The government has certain rights in the invention.

### FIELD

This disclosure concerns a recombinant, attenuated mumps virus vaccine based on the Jeryl Lynn (JL) strain. The genotype A JL strain is modified to replace the fusion (F) and hemagglutinin-neuraminidase (HN) genes with F and HN genes of genotype G mumps viruses.

### BACKGROUND

The Jeryl Lynn mumps virus vaccine strain is the only mumps vaccine used in the U.S. and the predominant strain used globally. Vaccine use has been responsible for the near elimination of mumps in those countries that include it in their childhood immunization schedule. However, since 2004, there has been a resurgence in cases globally, including in the U.S., which in 2006 experienced its largest mumps outbreak in over 20 years. Unusually large mumps outbreaks continue to occur, despite widespread vaccination.

Previous studies have linked disease resurgence with the appearance of a new mumps virus genotype, defined based on nucleotide sequence variation within the viral small hydrophobic (SH) gene. Whereas the Jeryl Lynn vaccine strain is a genotype A virus, the strains responsible for the global resurgence of disease are genotype G viruses. Although mumps virus is considered to be serologically monotypic (antibodies produced against one mumps virus strain should effectively recognize all other mumps virus strains), there is evidence of significant antigenic differences between genotype A and G viruses, resulting in a reduced capacity of antibodies induced by the Jeryl Lynn vaccine to neutralize genotype G strains (Rubin et al., J Virol 86:615-620, 2012; Rubin et al., J Infect Dis 198: 508-515, 2008). Further complicating the issue is the natural tendency for antibody titers to decline over time. Studies have found that although shortly after vaccination only 2-3% of Jeryl Lynn vaccinees lacked adequate levels of neutralizing antibody against genotype A or G viruses, by 10 years post-vaccination, approximately 10% of vaccinees lacked adequate levels of neutralizing antibody against genotype A viruses and approximately 30% lacked adequate levels of neutralizing antibody against genotype G viruses (Cortese et al., J Infect Dis 204: 1413-1422, 2011; Rubin et al., J Infect Dis 198: 508-515, 2008; Date et al., J Infect Dis 197: 1662-1668, 2008). This is consistent with the observation that although mumps was historically a disease of childhood, outbreaks now occur predominately among persons 18-24 years of age. These findings suggest that disease resurgence is due to the combination of waning immunity and antigenic differences between the vaccine genotype and the circulating genotype. Thus, a need exists for the development of new, more efficacious mumps virus vaccines.

### SUMMARY

Recombinant, attenuated mumps viruses are described herein. The recombinant viruses are based on the genotype A Jeryl Lynn vaccine strain, but are modified to express genotype G fusion (F) and hemagglutinin-neuraminidase (HN) proteins encoded by genotype G viruses. The recombinant viruses optionally include one or more mutations (such as one, two, three or more) that prevent expression of viral protein V. The recombinant mumps virus can be used as a vaccine to inhibit mumps virus infection and the development of mumps disease.

Provided herein is an isolated nucleic acid molecule comprising a cDNA sequence encoding a mumps virus nucleoprotein (N) gene, a mumps virus phosphoprotein (P) gene (which encodes the mumps virus V, P and I proteins), a mumps virus matrix protein (M) gene, a mumps virus fusion protein (F) gene, a mumps virus small hydrophobic protein (SH) gene, a mumps virus hemagglutinin-neuraminidase protein (HN) gene and a mumps virus large protein (L) gene, wherein the N, P, M, SH and L gene sequences are based on Jeryl Lynn strain gene sequences, and the F and HN genes are based on genotype G gene sequences. In some embodiments, the P gene comprises at least one mutation that prevents expression of the V protein. Plasmids that include an isolated nucleic acid molecule disclosed herein, and isolated host cells comprising a disclosed plasmid, are further provided.

Also provided are recombinant mumps viruses, wherein the genome of the recombinant mumps viruses comprise an N gene, a P gene, an M gene, an F gene, an SH gene, an HN gene and a L gene, wherein the N, P, M, SH and L genes were synthesized based on Jeryl Lynn strain sequences, and the F and HN genes were synthesized based on genotype G sequences. In some embodiments, the P gene includes at least one mutation that prevents expression of the V protein. Compositions that include a recombinant mumps virus disclosed herein and a pharmaceutically acceptable carrier are also provided.

Further provided is a method of eliciting an immune response against mumps virus in a subject by administering to the subject an effective amount of a recombinant mumps virus or composition disclosed herein. Methods of immunization a subject against mumps virus by administering an effective amount of a recombinant mumps virus or composition disclosed herein are also provided.

Also provided is a collection of plasmids that can be used to rescue a recombinant mumps virus disclosed herein. In some embodiments, the collection of plasmids includes a plasmid comprising a cDNA sequence encoding a recombinant mumps virus genome disclosed herein, a plasmid comprising a mumps virus N gene ORF, a plasmid comprising a mumps virus P gene ORF, and a plasmid comprising a mumps virus L gene ORF. Further provided is a method of producing a recombinant mumps virus. In some embodiments, the method includes transfecting cultured cells with a collection of plasmids disclosed herein; incubating the transfected cells for a sufficient time to allow for mumps virus replication; and collecting the recombinant mumps virus from the cell culture supernatant.

The foregoing and other objects, features, and advantages of the disclosure will become more apparent from the following detailed description, which proceeds with reference to the accompanying figures.

### BRIEF DESCRIPTION OF THE DRAWINGS

FIGS. 1A-1H are schematics of the seven fragments used for cloning the full-length JL strain mumps virus to produce plasmid pJL.

FIG. 2 is a vector map of plasmid pJL. Fragments 1, 2, 3, 4/5, 4, 5, 6 and 7 are indicated.

FIGS. 3A-3C are schematics of nucleic acid fragments for inserting genotype G fusion (F) and hemagglutinin-neuraminidase (HN) genes into pJL.

FIG. 4 is a schematic of the pUC57-Kan vector containing the JL N and V genes.

FIG. 5 is a graph comparing neurovirulence scores of several approved mumps vaccines, RecombiMumps-G(dV), and several wild-type mumps virus isolates. Merck: Merck Jeryl Lynn vaccine (Genotype A); GSK: GlaxoSmithKline RIT-4385 vaccine (Genotype A); JL-Clone: FDA produced molecular clone of “Merck” (Genotype A); JL(G-F/HN): RecombiMumps-G(dV) precursor (before V mutation and passage); Urabe-AM9: Urabe-AM9 vaccine strain (Genotype B); WT-TN: Wild type clinical isolate from Tennessee (Genotype G); WT-IA: Wild type clinical isolate from Iowa (Genotype G); WT-Lol: Wild type clinical isolate from London (Genotype D); Urabe-CI: Clinical isolate, Urabe-AM9 meningitis (Genotype B); WT-NY: Wild type clinical isolate from New York (Genotype G); WT-Petrenko: Wild type clinical isolate from Russia (Genotype H); and WT-88-1961: Wild type clinical isolate from New York (Genotype H).

FIG. 6 is a schematic of the mumps virus negative-sense RNA genome. The viral genome includes the N, P, M, F, SH, HN and L genes, each encoding a single protein, with the exception of the P gene, which encodes three proteins (V, P and I).

FIGS. 7A-7C are graphs showing neutralizing antibody titers following immunization of monkeys with JL (n=5) or RecombiMumps-G(dV) (n=5). Rhesus macaques were immunized intramuscularly with 1.6×10⁵ pfu of JL or RecombiMumps-G(dV) and boosted with the same dose 30 days later. Sera were collected before vaccination (day 0) and at approximately days 15, 30, 45 and 60 and tested for their ability to neutralize JL (FIG. 7A), RecombiMumps-G(dV) (FIG. 7B) or a wild-type genotype G mumps virus (Iowa-G; FIG. 7C). In each graph, lines 1-5 represent the five monkeys vaccinated with JL and lines 6-10 represent the five monkeys vaccinated with RecombiMumps-G(dV). The inflection point (day 30) represents administration of the second dose (booster dose).

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on Dec. 6, 2019, 48.4 KB, which is incorporated by reference herein. In the accompanying sequence listing:

SEQ ID NO: 1 is the nucleotide sequence of the RecombiMumps-G(dV) genome having the following features:

nucleotides 146-1795—N gene (Jeryl Lynn)

nucleotides 1832-1835—mutations to introduce restriction site 1

nucleotides 1979-3152—P gene, encoding V, P and I proteins (Jeryl Lynn)

nucleotides 2459-2461—stop codon 1

nucleotides 2460 and 2462—mutations to introduce stop codon 1

nucleotides 2495—mutation to introduce stop codon 2

nucleotides 2495-2497—stop codon 2

nucleotide 2516—mutation to introduce stop codon 3

nucleotides 2516-2518—stop codon 3

nucleotides 3264-4380—M gene (Jeryl Lynn)

nucleotides 4463, 4466 and 4469—mutations to introduce restriction site 2

nucleotides 4546-6162—F gene (genotype G)

nucleotide 6020—mutation

nucleotides 6268-6441—SH gene (Jeryl Lynn)

nucleotides 6466 and 6472-6473—mutations to introduce restriction site 3

nucleotides 6614-8362—HN gene (genotype G)

nucleotides 8397 and 8399—mutations to introduce restriction site 4

nucleotides 8438-15223—L gene (Jeryl Lynn)

nucleotide 9634—mutation to introduce restriction site 5

SEQ ID NO: 2 is the nucleotide sequence of a mumps virus genotype G consensus F gene.

SEQ ID NO: 3 is the nucleotide sequence of a mumps virus genotype G consensus HN gene.

SEQ ID NO: 4 is a nucleotide sequence for cloning of the consensus F gene sequence having the following features:

nucleotides 1-205—upstream JL sequence modified to include a PmeI site

nucleotides 22-29—PmeI site

nucleotides 106-1722—genotype G mumps virus consensus F ORF

nucleotides 1723-2051—downstream JL sequence modified to include restriction sites

nucleotides 2026-2033—AsiSI site

nucleotides 2041-2046—MluI site

nucleotides 2045-2050—SalI site.

SEQ ID NO: 5 is a nucleotide sequence for cloning of the consensus HN gene sequence having the following features:

nucleotides 1-162—upstream JL sequence modified to include restriction sites

nucleotides 1-7—NotI site

nucleotides 15-22—AsiSI site

nucleotides 163-1911—genotype G mumps virus consensus HN ORF

nucleotides 1912-1967—downstream JL sequence modified to include restriction sites

nucleotides 1942-1949—AscI site

nucleotides 1957-1962—KpnI site

nucleotides 1962-1967—XhoI site.

SEQ ID NO: 6 is the nucleotide sequence of a portion of the V gene modified to include three non-native stop codons having the following features:

nucleotides 1-6—a native ClaI site

nucleotides 72-74—non-native stop codon

nucleotides 108-110—non-native stop codon

nucleotides 129-131—non-native stop codon

nucleotides 437-443—a native EcoO109I site.

SEQ ID NO: 7 is the nucleotide sequence of the N gene ORF modified to include restriction sites for cloning of helper plasmid pTM1-N having the following features:

nucleotides 1-27—overhang sequence containing a NcoI site

nucleotides 19-24—NcoI site

nucleotides 28-1677—N gene ORF

nucleotides 1678-1737—overhang sequence containing an XhoI site

nucleotides 1714-1719—XhoI site.

SEQ ID NO: 8 is the nucleotide sequence of the P gene ORF modified to include restriction sites for cloning of helper plasmid pTM1-P having the following features:

nucleotides 1-20—overhang sequence

nucleotides 19-24—an NcoI site

nucleotides 21-1196—P gene ORF

nucleotides 481-482—insertion of two guanine (“gg”) nucleotides

nucleotides 1197-1281—overhang sequence containing an XhoI site

nucleotides 1258-1263—XhoI site.

SEQ ID NO: 9 is the nucleotide sequence of the L gene ORF modified to include restrictions sites for cloning of helper plasmid pTM1-L having the following features:

nucleotides 1-45—upstream sequence containing an AscI site

nucleotides 1-8—AscI site

nucleotides 46-6831—L gene ORF

nucleotides 3527-3532—an AatII site

nucleotides 6581-6586—a NcoI site

nucleotides 6832-6899—downstream sequence containing a PstI site

nucleotides 6893-6899—PstI site.

### DETAILED DESCRIPTION

### I. Abbreviations

CIP calf intestinal phosphatase

F fusion protein

HN hemagglutinin-neuraminidase protein

JL Jeryl Lynn

L large protein

M matrix protein

MuV mumps virus

N nucleoprotein

ORF open reading frame

P phosphoprotein

SH small hydrophobic protein

### II. Terms and Methods

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the term “comprises” means “includes.” Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

Adjuvant: A substance or vehicle that non-specifically enhances the immune response to an antigen (for example, mumps virus antigen). Adjuvants can be used with the compositions disclosed herein, for example as part of a pharmaceutical mumps virus vaccine composition provided herein. Adjuvants can include a suspension of minerals (alum, aluminum hydroxide, or phosphate) on which antigen is adsorbed; or water-in-oil emulsion in which antigen solution is emulsified in mineral oil (for example, Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants also include biological molecules, such as costimulatory molecules. Exemplary biological adjuvants include IL-2, RANTES, GM-CSF, TNF-α, IFN-γ, G-CSF, LFA-3, CD72, B7-1, B7-2, OX-40L and 41 BBL. In one example the adjuvant is one or more toll-like receptor (TLR) agonists, such as an agonist of TLR1/2 (which can be a synthetic ligand) (for example, Pam3Cys), TLR2 (for example, CFA, Pam2Cys), TLR3 (for example, polyL:C, poly A:U), TLR4 (for example, MPLA, Lipid A, and LPS), TLR5 (for example, flagellin), TLR7 (for example, gardiquimod, imiquimod, loxoribine, Resiquimod®), TLR7/8 (for example, R0848), TLR8 (for example, imidazoquionolines, ssPolyU, 3M-012), TLR9 (for example, ODN 1826 (type B), ODN 2216 (type A), CpG oligonucleotides) and/or TLR11/12 (for example, profilin). In one example, the adjuvant is lipid A, such as lipid A monophosphoryl (MPL) from Salmonella enterica serotype Minnesota Re 595 (for example, Sigma Aldrich Catalog #L6895).

Administer: As used herein, administering a composition (such as one containing a mumps virus composition) to a subject means to give, apply or bring the composition into contact with the subject. Administration can be accomplished by any of a number of routes, such as, for example, intramuscular, intranasal, pulmonary, topical, oral, subcutaneous, intraperitoneal, intravenous, intrathecal, rectal, vaginal and intradermal.

Antigen or immunogen: A compound, composition, or substance that can stimulate the production of antibodies or a T-cell response in an animal, including compositions that are injected or absorbed into an animal. An antigen reacts with the products of specific humoral or cellular immunity, including those induced by heterologous immunogens.

Attenuated: In the context of the type of live virus described herein, the virus is attenuated if its ability to produce disease is reduced (for example, eliminated) compared to a wild-type virus. In some embodiments, the ability of an attenuated virus to cause disease in a subject is reduced at least about 10%, at least about 25%, at least about 50%, at least about 75% or at least about 90% relative to wild-type virus.

Collection: A group or combination of items. In the context of the present disclosure, a “collection of plasmids” is a group of plasmids that are used together, such as to rescue a recombinant mumps virus.

Heterologous: A heterologous protein or nucleic acid refers to a protein or nucleic acid derived from a different source or species.

Immune response: A response of a cell of the immune system, such as a B-cell, T-cell, macrophage or polymorphonucleocyte, to a stimulus such as an antigen/immunogen or vaccine (such as a mumps virus vaccine). An immune response can include any cell of the body involved in a host defense response, including for example, an epithelial cell that secretes an interferon or a cytokine. An immune response includes, but is not limited to, an innate immune response or inflammation. As used herein, a protective immune response refers to an immune response that protects a subject from infection (prevents infection or prevents the development of disease associated with infection). Methods of measuring immune responses include, for example, measuring proliferation and/or activity of lymphocytes (such as B or T cells), secretion of cytokines or chemokines, inflammation, antibody production and the like.

Immunize: To render a subject (such as a mammal) protected from an infectious disease (for example, mumps), such as by vaccination.

Isolated: An “isolated” biological component (such as a nucleic acid, protein, or virus) has been substantially separated or purified away from other biological components (such as cell debris, or other proteins or nucleic acids). Biological components that have been “isolated” include those components purified by standard purification methods. The term also embraces recombinant nucleic acids, proteins, viruses, as well as chemically synthesized nucleic acids or peptides.

Modification: A change in a nucleic acid or protein sequence. For example, sequence modifications include, for example, substitutions, insertions and deletions, or combinations thereof. For proteins, insertions include amino and/or carboxyl terminal fusions as well as intrasequence insertions of single or multiple amino acid residues. Insertions for nucleic acid sequence include 5′ or 3′ additions or intrasequence insertions of single or multiple nucleotides. Deletions are characterized by the removal of one or more amino acid residues from a protein sequence or one or more nucleotides from a nucleic acid sequence. Substitutional modifications are those in which at least one amino acid residue or nucleotide has been removed and a different residue or nucleotide inserted in its place. Substitutions, deletions, insertions or any combination thereof may be combined to arrive at a final mutant sequence. Protein modifications can be prepared, for example, by modification of nucleotides in the DNA encoding the protein, thereby producing DNA encoding the modification. A “modified” protein, nucleic acid or virus is one that has one or more modifications as outlined above.

Mumps: An infectious disease caused by mumps virus. Mumps is characterized by inflammation of the salivary glands, typically the parotid glands. Severe complications of mumps virus infection can occur, such as meningitis, encephalitis, pancreatitis, oophoritis (in females), orchitis (in males) and hearing loss.

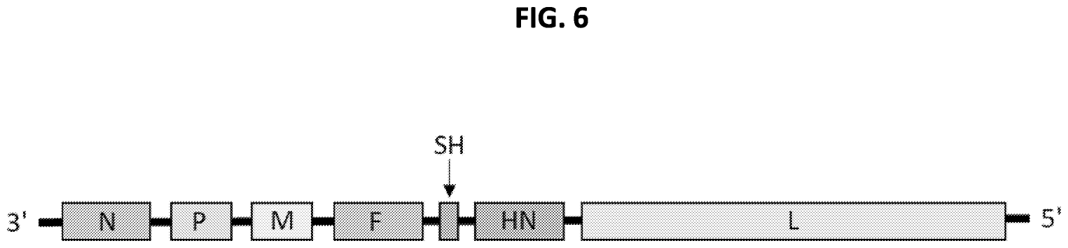

Mumps virus (MuV): A non-segmented, negative-stranded RNA virus of the family Paramyxoviridae, subfamily Paramyxovirinae, genus Rubulavirus that causes mumps disease. Mumps virions are pleomorphic particles ranging in size from 100 to 800 nm (McCharthy et al., J Gen Virol 48:395-399, 1980), and consist of a helical ribonucleocapsid core surrounded by a host cell-derived lipid envelope. Mumps virus genomic RNA contains 7 tandemly linked transcription units that encode open reading frames for the nucleoprotein (N), phosphoprotein (P), V protein, I protein, matrix (M) protein, fusion (F) protein, small hydrophobic (SH) protein, hemagglutinin-neuraminidase (HN) protein, and the large (L) protein (Carbone and Rubin, In: Knipe D M, Howley P M, editors. Fields Virology. Philadelphia: Wolters Kluwer Health/Lippincott Williams & Wilkins; pp. 1528-1530, 2007). A schematic of the mumps virus genome is shown in FIG. 6. Due to RNA editing by insertion of guanine nucleotides, the P gene (also referred to as the “V/P/I gene”) results in three mRNA transcripts corresponding to the V, P and I proteins (Paterson and Lamb, J Virol 64:4137-4145, 1990). Specifically, faithful transcription of the P gene produces the V protein, insertion of two guanine nucleotides produces an mRNA encoding the P protein, and insertion of four guanine residues results in an mRNA encoding the I protein (Sauder et al., J Virol 85(14): 7059-7069, 2011). The SH gene is the most variable gene amongst different genotypes of MuV and is therefore generally used as the basis for genotyping. There are 12 known genotypes of MuV, designated as genotypes A, B, C, D, F, G, H, I, J, K, L and N, that are currently circulating globally (Cui et al., PLoS One e0169561, 2017). Recent outbreaks of MuV have been caused by genotype G viruses in the Western hemisphere, genotypes J and F in the Asia-Pacific region and genotype H in the Middle East. Most current MuV vaccines are based on genotype A (Jeryl Lynn), genotype B (Urabe-AM9) or undetermined genotype (Leningrad-Zagreb) viruses (Rubin et al., J Virol 86(1): 615-620, 2011).

Nucleic acid molecule: A polymeric form of nucleotides, which may include both sense and anti-sense strands of RNA, cDNA, genomic DNA, genomic RNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A polynucleotide may include either or both naturally occurring and modified nucleotides linked together by naturally occurring and/or non-naturally occurring nucleotide linkages. “cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (such as rRNA, tRNA and mRNA) or a defined sequence of amino acids and the biological properties resulting therefrom.

ORF (open reading frame): A series of nucleotide triplets (codons) coding for amino acids without any termination codons. These sequences are usually translatable into a peptide.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington's Pharmaceutical Sciences, by E. W. Martin, Mack Publishing Co., Easton, Pa., 19th Edition, 1995, describes compositions and formulations suitable for pharmaceutical delivery of the disclosed compositions.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions (such as immunogenic compositions) to be administered can contain minor amounts of non-toxic auxiliary substances, such as wetting or emulsifying agents, preservatives, and pH buffering agents and the like, for example sodium acetate or sorbitan monolaurate. In particular embodiments, suitable for administration to a subject the carrier may be sterile, and/or suspended or otherwise contained in a unit dosage form containing one or more measured doses of the composition suitable to induce the desired immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

Plasmid: A circular nucleic acid molecule capable of autonomous replication in a host cell.

Polypeptide: Any chain of amino acids, regardless of length or post-translational modification (for example, glycosylation or phosphorylation). “Polypeptide” applies to amino acid polymers including naturally occurring amino acid polymers and non-naturally occurring amino acid polymer as well as in which one or more amino acid residue is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Promoter: A promoter is an array of nucleic acid control sequences that direct transcription of a nucleic acid. A promoter includes necessary nucleic acid sequences near the start site of transcription. A promoter also optionally includes distal enhancer or repressor elements. A “constitutive promoter” is a promoter that is continuously active and is not subject to regulation by external signals or molecules. In contrast, the activity of an “inducible promoter” is regulated by an external signal or molecule (for example, a transcription factor). In one embodiment, the promoter is a T7 promoter (from bacteriophage T7).

Purified: The term “purified” does not require absolute purity; rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to fractionation to remove various components of the initial preparation, such as proteins, cellular debris, and other components.

Recombinant: A recombinant nucleic acid molecule is one that has a sequence that is not naturally occurring, for example, includes one or more nucleic acid substitutions, deletions or insertions, and/or has a sequence that is made by an artificial combination of two otherwise separated segments of sequence. This artificial combination can be accomplished by chemical synthesis or by the artificial manipulation of isolated segments of nucleic acids, for example, by genetic engineering techniques. A recombinant virus is one that includes a genome that includes a recombinant nucleic acid molecule. A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell, or into the genome of a recombinant virus.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a given gene or protein will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237-244, 1988; Higgins and Sharp, CABIOS 5:151-153, 1989; Corpet et al., Nucleic Acids Research 16:10881-10890, 1988; and Pearson and Lipman, Proc. Natl. Acad. Sci. U.S.A. 85:2444, 1988. Altschul et al., Nature Genet. 6:119-129, 1994.

The NCBI Basic Local Alignment Search Tool (BLAST™) (Altschul et al., J. Mol. Biol. 215:403-410, 1990) is available from several sources, including the National Center for Biotechnology Information (NCBI, Bethesda, Md.) and on the Internet, for use in connection with the sequence analysis programs blastp, blastn, blastx, tblastn and tblastx.

Subject: Living multi-cellular vertebrate organisms, a category that includes both human and non-human mammals. In some examples, a subject is one that can be infected with mumps virus, such as humans.

Synthetic: Produced by artificial means in a laboratory, for example a synthetic nucleic acid or protein can be chemically synthesized in a laboratory.

Therapeutically effective amount: The amount of agent, such as a disclosed recombinant mumps virus, that is sufficient to prevent, treat (including prophylaxis), reduce and/or ameliorate the symptoms and/or underlying causes of a disorder or disease, for example to prevent, inhibit, and/or treat mumps virus infection and/or mumps disease. In some embodiments, a therapeutically effective amount is sufficient to reduce or eliminate a symptom of a disease. For instance, this can be the amount necessary to inhibit or prevent viral replication or to measurably alter outward symptoms of the viral infection.

In one example, a desired response is to inhibit or reduce or prevent mumps virus infection. The MuV infection does not need to be completely eliminated or reduced or prevented for the method to be effective. For example, administration of a therapeutically effective amount of the agent can decrease the MuV infection (for example, as measured by infection of cells, or by number or percentage of subjects infected by MuV) by a desired amount, for example by at least 50%, at least 60%, at least 70%, at least 80%, at least 90%, at least 95%, at least 98%, or even at least 100% (elimination or prevention of detectable MuV infection), as compared to a suitable control.

It is understood that obtaining a protective immune response against a pathogen can require multiple administrations of the immunogenic composition. Thus, a therapeutically effective amount encompasses a fractional dose that contributes in combination with previous or subsequent administrations to attaining a protective immune response. For example, a therapeutically effective amount of an agent can be administered in a single dose, or in several doses, for example daily, during a course of treatment (such as a prime-boost vaccination treatment). However, the therapeutically effective amount can depend on the subject being treated, the severity and type of the condition being treated, and the manner of administration. A unit dosage form of the agent can be packaged in a therapeutic amount, or in multiples of the therapeutic amount, for example, in a vial (such as with a pierceable lid) or syringe having sterile components.

Transformed: A transformed cell is a cell into which has been introduced a nucleic acid molecule by molecular biology techniques. As used herein, the term transformation encompasses all techniques by which a nucleic acid molecule might be introduced into such a cell, including transfection with viral vectors, transformation with plasmid vectors, and introduction of naked DNA by electroporation, lipofection, and particle gun acceleration.

Vaccine: A preparation of immunogenic material capable of stimulating an immune response, administered for the prevention, amelioration, or treatment of infectious or other types of disease. The immunogenic material may include attenuated or killed microorganisms (such as attenuated viruses), or antigenic proteins, peptides or DNA derived from them. Vaccines may elicit both prophylactic (preventative) and therapeutic responses. Methods of administration vary according to the vaccine, but may include inoculation, ingestion, inhalation or other forms of administration. Inoculations can be delivered by any of a number of routes, including parenteral, such as intravenous, subcutaneous or intramuscular. Vaccines may be administered with an adjuvant to boost the immune response.

### III. Overview of Several Embodiments

The present disclosure describes recombinant, attenuated mumps viruses, such as for use as vaccines for protection against mumps virus infection and the development of mumps disease. The recombinant viruses are based on the genotype A Jeryl Lynn mumps virus vaccine strain, but are modified to express genotype G fusion (F) and hemagglutinin-neuraminidase (HN) proteins, such as genotype G consensus F and HN proteins. The recombinant viruses optionally include one or more mutations that prevent expression of viral protein V, which is encoded by the P gene.

Provided herein is an isolated nucleic acid molecule comprising a cDNA sequence encoding a mumps virus nucleoprotein (N) gene, a mumps virus phosphoprotein (P) gene (which encodes the V, P and I proteins), a mumps virus matrix protein (M) gene, a mumps virus F gene, a mumps virus small hydrophobic protein (SH) gene, a mumps virus HN gene and a mumps virus large protein (L) gene, wherein the N, P, M, SH and L gene sequences are based on Jeryl Lynn strain gene sequences, and the F and HN gene sequences are based on genotype G mumps virus gene sequences. In some embodiments, the F gene is a synthetic gene comprising a consensus genotype G mumps virus F gene sequence and/or the HN gene is a synthetic gene comprises a consensus genotype G mumps virus HN gene sequence, or both. In other embodiments, the F gene sequence is based on a native genotype G mumps virus F gene sequence and/or the HN gene is based on a native genotype G mumps virus HN gene sequence. For example, the genotype G mumps virus F gene sequence or HN gene sequence can be at least 80%, at least 85%, at least 90%, at least 95%, at least 96%, at least 97%, at least 98%, at least 99% or 100% identical to a wild-type F or HN gene sequence from a particular genotype G mumps virus strain.

In some embodiments, the P gene includes at least one mutation that prevents expression of the mumps virus V protein. In some examples, the at least one mutation introduces one or more stop codons in the V protein open reading frame (ORF), such as one, two, three, four or five stop codons. In some examples, the at least one mutation does not prevent expression of the mumps virus I protein or the P protein. In particular examples, the at least one mutation includes one, two or three stop codons at nucleotides 2459-2461, nucleotides 2495-2497 and/or nucleotides 2516-2518 of a recombinant mumps virus genome, numbered with reference to SEQ ID NO: 1.

In some embodiments, the sequence of the N gene is at least 80%, at least 85%, at least 90%, at least 95%, at least 96%, at least 97%, at least 98%, at least 99% identical to nucleotides 146-1795 of SEQ ID NO: 1. In some examples, the sequence of the N gene comprises nucleotides 146-1795 of SEQ ID NO: 1.

In some embodiments, the sequence of the P gene is at least 80%, at least 85%, at least 90%, at least 95%, at least 96%, at least 97%, at least 98%, at least 99% identical to nucleotides 1979-3152 of SEQ ID NO: 1. In some examples, the sequence of the P gene comprises nucleotides 1979-3152 of SEQ ID NO: 1.

In some embodiments, the sequence of the M gene is at least 80%, at least 85%, at least 90%, at least 95%, at least 96%, at least 97%, at least 98%, at least 99% identical to nucleotides 3264-4380 of SEQ ID NO: 1. In some examples, the sequence of the M gene comprises nucleotides 3264-4380 of SEQ ID NO: 1.

In some embodiments, the sequence of the SH gene is at least 80%, at least 85%, at least 90%, at least 95%, at least 96%, at least 97%, at least 98%, at least 99% identical to nucleotides 6268-6441 of SEQ ID NO: 1. In some examples, the sequence of the SH gene comprises nucleotides 6268-6441 of SEQ ID NO: 1.

In some embodiments, the sequence of the L gene is at least 80%, at least 85%, at least 90%, at least 95%, at least 96%, at least 97%, at least 98%, at least 99% identical to nucleotides 8438-15223 of SEQ ID NO: 1. In some examples, the sequence of the L gene comprises nucleotides 8438-15223 of SEQ ID NO: 1.

In some embodiments, the sequence of the F gene is at least 80%, at least 85%, at least 90%, at least 95%, at least 96%, at least 97%, at least 98%, at least 99% identical to SEQ ID NO: 2. In some examples, the sequence of the F gene comprises SEQ ID NO: 2.

In some embodiments, the sequence of the HN gene is at least 80%, at least 85%, at least 90%, at least 95%, at least 96%, at least 97%, at least 98%, at least 99% identical to SEQ ID NO: 3. In some examples, the sequence of the HN gene comprises SEQ ID NO: 3.

In some embodiments, the sequence of the nucleic acid molecule is at least 80%, at least 85%, at least 90%, at least 95%, at least 96%, at least 97%, at least 98%, at least 99% identical to SEQ ID NO: 1. In some examples, the sequence of the nucleic acid molecule comprises or consists of SEQ ID NO: 1.

Also provided herein is a plasmid that includes an isolated nucleic acid molecule disclosed herein. In some embodiments, the plasmid further includes a heterologous promoter. In particular examples, the promoter is a T7 promoter. Further provided are isolated host cells that include a plasmid disclosed herein. In some examples, the host cells are permissive for mumps virus infection. In particular examples, the host cells express T7 polymerase. Also provided herein are recombinant mumps viruses, wherein the genome of the recombinant mumps virus is encoded by a nucleic acid molecule or plasmid disclosed herein.

Further provided herein are recombinant mumps viruses, wherein the genome of the recombinant mumps virus comprises a nucleoprotein (N) gene, a phosphoprotein (P) gene, a matrix protein (M) gene, a fusion protein (F) gene, a small hydrophobic protein (SH) gene, a hemagglutinin-neuraminidase protein (HN) gene and a large protein (L) gene, wherein the N, P, M, SH and L genes are Jeryl Lynn strain mumps virus genes, and the F and HN genes are genotype G mumps virus F and HN genes.

In some embodiments, the F gene of the recombinant mumps virus genome is a synthetic gene comprising a consensus genotype G mumps virus F gene sequence and/or the HN gene of the recombinant mumps virus genome is a synthetic gene comprises a consensus genotype G mumps virus HN gene sequence, or both. In other embodiments, the F gene of the recombinant virus genome is a native genotype G mumps virus F gene and/or the HN gene of the recombinant mumps virus genome is a native genotype G mumps virus HN gene. For example, the native genotype G mumps virus F gene or HN gene can be at least 80%, at least 85%, at least 90%, at least 95%, at least 96%, at least 97%, at least 98%, at least 99% or 100% identical to a wild-type F or HN gene from a particular genotype G mumps virus strain.

In some embodiments, the P gene of the recombinant mumps virus genome includes at least one mutation that prevents expression of the mumps virus V protein. In some examples, the at least one mutation introduces one or more stop codons in the V protein ORF, such as one, two, three, four or five stop codons. In some examples, the at least one mutation does not prevent expression of the mumps virus I protein or the P protein. In particular examples, the at least one mutation includes one, two or three stop codons at nucleotides 2459-2461, nucleotides 2495-2497 and/or nucleotides 2516-2518 of a recombinant mumps virus genome, numbered with reference to SEQ ID NO: 1.

In some embodiments, the sequence of the N gene of the recombinant mumps virus genome is at least 80%, at least 85%, at least 90%, at least 95%, at least 96%, at least 97%, at least 98%, at least 99% identical to nucleotides 146-1795 of SEQ ID NO: 1. In some examples, the sequence of the N gene comprises nucleotides 146-1795 of SEQ ID NO: 1.

In some embodiments, the sequence of the P gene of the recombinant mumps virus genome is at least 80%, at least 85%, at least 90%, at least 95%, at least 96%, at least 97%, at least 98%, at least 99% identical to nucleotides 1979-3152 of SEQ ID NO: 1. In some examples, the sequence of the P gene comprises nucleotides 1979-3152 of SEQ ID NO: 1.

In some embodiments, the sequence of the M gene of the recombinant mumps virus genome is at least 80%, at least 85%, at least 90%, at least 95%, at least 96%, at least 97%, at least 98%, at least 99% identical to nucleotides 3264-4380 of SEQ ID NO: 1. In some examples, the sequence of the M gene comprises nucleotides 3264-4380 of SEQ ID NO: 1.

In some embodiments, the sequence of the SH gene of the recombinant mumps virus genome is at least 80%, at least 85%, at least 90%, at least 95%, at least 96%, at least 97%, at least 98%, at least 99% identical to nucleotides 6268-6441 of SEQ ID NO: 1. In some examples, the sequence of the SH gene comprises nucleotides 6268-6441 of SEQ ID NO: 1.

In some embodiments, the sequence of the L gene of the recombinant mumps virus genome is at least 80%, at least 85%, at least 90%, at least 95%, at least 96%, at least 97%, at least 98%, at least 99% identical to nucleotides 8438-15223 of SEQ ID NO: 1. In some examples, the sequence of the L gene comprises nucleotides 8438-15223 of SEQ ID NO: 1.

In some embodiments, the sequence of the F gene of the recombinant mumps virus genome is at least 80%, at least 85%, at least 90%, at least 95%, at least 96%, at least 97%, at least 98%, at least 99% identical to SEQ ID NO: 2. In some examples, the sequence of the F gene comprises SEQ ID NO: 2.

In some embodiments, the sequence of the HN gene of the recombinant mumps virus genome is at least 80%, at least 85%, at least 90%, at least 95%, at least 96%, at least 97%, at least 98%, at least 99% identical to SEQ ID NO: 3. In some examples, the sequence of the HN gene comprises SEQ ID NO: 3.

In some embodiments, the sequence of the recombinant mumps virus genome is at least 80%, at least 85%, at least 90%, at least 95%, at least 96%, at least 97%, at least 98%, at least 99% identical to SEQ ID NO: 1. In some examples, the sequence of the recombinant mumps virus genome comprises or consists of SEQ ID NO: 1.

Also provided herein are compositions that include a recombinant mumps virus disclosed herein and a pharmaceutically acceptable carrier. In some embodiments, the composition further includes an adjuvant.

Further provided herein is a method of eliciting an immune response against mumps virus in a subject. In some embodiments, the method includes administering to the subject an effective amount of a recombinant mumps virus or composition disclosed here. In some embodiments, the recombinant mumps virus or composition is administered in a single dose. In other embodiments, the recombinant mumps virus or composition is administered in multiple doses, such as two, three, four or five doses.

Also provided herein is a collection of plasmids that can be used to rescue a recombinant mumps virus disclosed herein. In some embodiments, the collection of plasmids includes a plasmid comprising a cDNA sequence encoding a recombinant mumps virus genome disclosed herein (such as the genome of SEQ ID NO: 1); a plasmid comprising a mumps virus N gene ORF; a plasmid comprising a mumps virus P gene ORF; and a plasmid comprising a mumps virus L gene ORF.

In some embodiments, the plasmid comprising the mumps virus N gene ORF comprises a nucleotide sequence at least 80%, at least 85%, at least 90%, at least 95%, at least 96%, at least 97%, at least 98%, at least 99% identical to SEQ ID NO: 7. In some examples, the plasmid comprises the nucleotide sequence of SEQ ID NO: 7.

In some embodiments, the plasmid comprising the mumps virus P gene ORF comprises a nucleotide sequence at least 80%, at least 85%, at least 90%, at least 95%, at least 96%, at least 97%, at least 98%, at least 99% identical to SEQ ID NO: 8. In some examples, the plasmid comprises the nucleotide sequence of SEQ ID NO: 8.

In some embodiments, the plasmid comprising the mumps virus L gene ORF comprises a nucleotide sequence at least 80%, at least 85%, at least 90%, at least 95%, at least 96%, at least 97%, at least 98%, at least 99% identical to SEQ ID NO: 9. In some examples, the plasmid comprises the nucleotide sequence of SEQ ID NO: 9.

In some embodiments, one or more (such as one, two, three or four) plasmids comprise a T7 polymerase promoter. In some embodiments, all four plasmids comprise a T7 promoter.

Further provided is a method of producing a recombinant mumps virus. In some embodiments, the method includes transfecting cultured cells with a collection of plasmids disclosed herein; incubating the transfected cells for a sufficient time to allow for mumps virus replication; and collecting the recombinant mumps virus from the cell culture supernatant. In some examples, the cultured cells express T7 polymerase.

### IV. Compositions

Compositions that include a recombinant mumps virus disclosed herein and a pharmaceutically acceptable carrier are provided. Such compositions can be administered to subjects by a variety of administration modes known to the person of ordinary skill in the art, for example, intramuscular, subcutaneous, intravenous, intra-arterial, intra-articular, intraperitoneal, or parenteral routes. Actual methods for preparing administrable compositions will be known or apparent to those skilled in the art and are described in more detail in such publications as Remington's Pharmaceutical Sciences, 19^{th} Ed., Mack Publishing Company, Easton, Pa., 1995.

Recombinant mumps viruses described herein can be formulated with pharmaceutically acceptable carriers to help retain biological activity while also promoting increased stability during storage within an acceptable temperature range. Potential carriers include, but are not limited to, physiologically balanced culture medium, phosphate buffered saline solution, water, emulsions (for example, oil/water or water/oil emulsions), various types of wetting agents, cryoprotective additives or stabilizers such as proteins, peptides or hydrolysates (for example, albumin, gelatin), sugars (for example, sucrose, lactose, sorbitol), amino acids (for example, sodium glutamate), or other protective agents. The resulting aqueous solutions may be packaged for use as is or lyophilized. Lyophilized preparations are combined with a sterile solution prior to administration for either single or multiple dosing.

Formulated compositions, especially liquid formulations, may contain a bacteriostat to prevent or minimize degradation during storage, including but not limited to effective concentrations (usually ≤1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The compositions of the disclosure can contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate.

The disclosed composition may optionally include an adjuvant to enhance an immune response of the host. Adjuvants, such as aluminum hydroxide (ALHYDROGEL®, available from Brenntag Biosector, Copenhagen, Denmark and Amphogel®, Wyeth Laboratories, Madison, N.J.), Freund's adjuvant, MPL™ (3-O-deacylated monophosphoryl lipid A; Corixa, Hamilton, Ind.), IL-12 (Genetics Institute, Cambridge, Mass.), TLR agonists (such as TLR-9 agonists), among many other suitable adjuvants well known in the art, can be included in the compositions. Suitable adjuvants are, for example, toll-like receptor agonists, alum, AlPO4, alhydrogel, Lipid-A and derivatives or variants thereof, oil-emulsions, saponins, neutral liposomes, liposomes containing the vaccine and cytokines, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, Ind.) and IL-12 (Genetics Institute, Cambridge, Mass.), among many other suitable adjuvants well known in the art, may be used as an adjuvant (Newman et al., 1998, Critical Reviews in Therapeutic Drug Carrier Systems 15:89-142). These adjuvants help to stimulate the immune system in a non-specific way, thus enhancing the immune response to a pharmaceutical product.

In some embodiments, the adjuvant is selected to elicit a ThI biased immune response in a subject.

In some instances, the adjuvant formulation includes a mineral salt, such as a calcium or aluminum (alum) salt, for example calcium phosphate, aluminum phosphate or aluminum hydroxide. In some embodiments, the adjuvant includes an oil and water emulsion, for example, an oil-in-water emulsion (such as MF59 (Novartis) or AS03 (GlaxoSmithKline). One example of an oil-in-water emulsion comprises a metabolizable oil, such as squalene, a tocol such as a tocopherol, for example, alpha-tocopherol, and a surfactant, such as sorbitan trioleate (Span 85) or polyoxyethylene sorbitan monooleate (Tween 80), in an aqueous carrier.

In some instances it may be desirable to combine a disclosed composition with other pharmaceutical products (for example, vaccines) which induce protective responses to other agents. For example, a composition including a recombinant mumps virus as described herein can be administered simultaneously or sequentially with other vaccines recommended by the Advisory Committee on Immunization Practices (ACIP; cdc.gov/vaccines/acip/index) for the targeted age group (for example, infants from approximately one to six months of age). As such, a disclosed composition described herein may be administered simultaneously or sequentially with vaccines against, for example, measles virus, rubella virus, varicella zoster virus, hepatitis B (HepB), diphtheria, tetanus and pertussis (DTaP), pneumococcal bacteria (PCV), Haemophilus influenzae type b (Hib), polio, influenza and rotavirus.

In some embodiments, the composition can be provided as a sterile composition. The composition typically contains an effective amount of a disclosed recombinant mumps virus and can be prepared by conventional techniques. Typically, the amount of recombinant virus in each dose of the composition is selected as an amount which induces an immune response without significant, adverse side effects. In some embodiments, the composition can be provided in unit dosage form for use to induce an immune response in a subject, for example, to prevent MuV infection in the subject. A unit dosage form contains a suitable single preselected dosage for administration to a subject, or suitable marked or measured multiples of two or more preselected unit dosages, and/or a metering mechanism for administering the unit dose or multiples thereof.

### V. Immunization Methods

The disclosed compositions can be administered to a subject to induce an immune response to mumps virus in a subject. In some embodiments, the subject is a human. The immune response can be a protective immune response, for example a response that prevents or reduces subsequent infection with mumps virus. Elicitation of the immune response can also be used to treat or inhibit mumps virus infection and illnesses associated therewith.

A subject can be selected for treatment that has, or is at risk for developing mumps virus infection, for example because of exposure or the possibility of exposure to mumps virus. Following administration of a disclosed composition, the subject can be monitored for mumps virus infection or symptoms associated therewith, or both.

Typical subjects intended for treatment with the compositions and methods of the present disclosure include humans, as well as any other animals susceptible to infection by a mumps virus. The compositions can be administered, for example, by beginning an immunization regimen anytime from 6 months to 12 months of age, or from 12 months to 15 months of age, or from 4 years to 6 years of age. In particular examples, a child is administered a first dose at 12-15 months of age and a second dose between 4-6 years of age. Booster doses at later ages can also be administered, such as if it is determined that the subject exhibits waning immunity against mumps virus. The Centers for Disease Control and Prevention recommends that teenagers and adults be current on mumps vaccination.

Administration of a disclosed recombinant mumps virus can be for prophylactic or therapeutic purpose. When provided prophylactically, the recombinant virus can be provided in advance of any symptom, for example in advance of infection. The prophylactic administration serves to prevent or ameliorate any subsequent infection. In some embodiments, the methods can involve selecting a subject at risk for contracting mumps virus infection, and administering a therapeutically effective amount of a disclosed recombinant mumps virus to the subject. The immunogen can be provided prior to the anticipated exposure to mumps virus so as to attenuate the anticipated severity, duration or extent of an infection and/or associated disease symptoms, after exposure or suspected exposure to the virus, or after the actual initiation of an infection. When provided therapeutically, the disclosed immunogens are provided at or after the onset of a symptom of mumps virus infection, or after diagnosis of mumps virus infection.

In some embodiments, administration of a disclosed recombinant mumps virus to a subject can elicit the production of an immune response that is protective against serious complications of mumps disease, such as encephalitis or meningitis, when the subject is subsequently infected or re-infected with a wild-type mumps virus. While the naturally circulating virus may still be capable of causing infection, there can be a reduced possibility of symptoms as a result of the vaccination and a possible boosting of resistance by subsequent infection by wild-type virus.

The recombinant mump viruses described herein, and compositions thereof, are provided to a subject in an amount effective to induce or enhance an immune response against mumps virus in the subject, such as a human. The actual dosage of disclosed virus will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimum prophylactic or therapeutic response.

A composition including one or more of the disclosed recombinant viruses can be used in coordinate (or prime-boost) vaccination protocols or combinatorial formulations. In certain embodiments, novel combinatorial immunogenic compositions and coordinate immunization protocols employ separate immunogens or formulations, each directed toward eliciting an anti-viral immune response, such as an immune response to mumps virus proteins. Separate immunogenic compositions that elicit the anti-viral immune response can be combined in a polyvalent immunogenic composition administered to a subject in a single immunization step, or they can be administered separately (in monovalent immunogenic compositions) in a coordinate (or prime-boost) immunization protocol.

There can be several boosts, and each boost can be a different disclosed immunogen. In some examples, the boost may be the same immunogen as another boost, or the prime. The prime and boost can be administered as a single dose or multiple doses, for example two doses, three doses, four doses, five doses, six doses or more can be administered to a subject over days, weeks or months. Multiple boosts can also be given, such one to five (for example, 1, 2, 3, 4 or 5 boosts), or more. Different dosages can be used in a series of sequential immunizations. For example a relatively large dose in a primary immunization and then a boost with relatively smaller doses.

In some embodiments, the boost can be administered about two, about three to eight, or about four, weeks following the prime, or several months after the prime. In some embodiments, the boost can be administered about 5, about 6, about 7, about 8, about 10, about 12, about 18, about 24, about 36, about 48 or about 50 months after the prime, or more or less time after the prime. Periodic additional boosts can also be used at appropriate time points to enhance the subject's “immune memory.” The adequacy of the vaccination parameters chosen, for example, formulation, dose, regimen and the like, can be determined by taking aliquots of serum from the subject and assaying antibody titers during the course of the immunization program. In addition, the clinical condition of the subject can be monitored for the desired effect, for example, prevention of mumps virus infection or improvement in disease state (for example, reduction in viral load). If such monitoring indicates that vaccination is sub-optimal, the subject can be boosted with an additional dose, and the vaccination parameters can be modified in a fashion expected to potentiate the immune response.

In some embodiments, each human dose comprises from about 3.0 log₁₀ to about 6.0 log₁₀ plaque forming units (“PFU”) or more of virus per patient, or from about 4.0 log₁₀ to 5.0 log₁₀ PFU virus per patient. The amount utilized in an immunogenic composition is selected based on the subject population (for example, infant or elderly). An optimal amount for a particular composition can be ascertained by standard studies involving observation of antibody titers and other responses in subjects. It is understood that a therapeutically effective amount of a disclosed recombinant mumps virus or composition thereof, can include an amount that is ineffective at eliciting an immune response by administration of a single dose, but that is effective upon administration of multiple dosages, for example in a prime-boost administration protocol.

Upon administration of a disclosed composition, the immune system of the subject typically responds to the immunogenic composition by producing antibodies specific for viral protein. Such a response signifies that an immunologically effective dose was delivered to the subject.

For each particular subject, specific dosage regimens can be evaluated and adjusted over time according to the individual need and professional judgment of the person administering or supervising the administration of the immunogenic composition. The dosage and number of doses will depend on the setting, for example, in an adult or anyone primed by prior mumps virus infection or immunization, a single dose may be a sufficient booster. In naïve subjects, in some examples, at least two doses would be given, for example, at least three doses. In some embodiments, an annual boost is given, for example, along with an annual influenza vaccination.

In some embodiments, the antibody response of a subject will be determined in the context of evaluating effective dosages/immunization protocols. In most instances it will be sufficient to assess the antibody titer in serum or plasma obtained from the subject. Decisions as to whether to administer booster inoculations and/or to change the amount of the therapeutic agent administered to the individual can be at least partially based on the antibody titer level. The antibody titer level can be based on, for example, an immunobinding assay which measures the concentration of antibodies in the serum which bind to a mumps virus antigen, such as mumps virus F protein or HN protein.

Determination of effective dosages is typically based on animal model studies followed up by human clinical trials and is guided by administration protocols that significantly reduce the occurrence or severity of targeted disease symptoms or conditions in the subject, or that induce a desired response in the subject (such as a neutralizing immune response). Suitable models in this regard include, for example, murine, rat, porcine, feline, ferret, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer a therapeutically effective amount of the composition (for example, amounts that are effective to elicit a desired immune response or alleviate one or more symptoms of a targeted disease). In alternative embodiments, an effective amount or effective dose of the composition may simply inhibit or enhance one or more selected biological activities correlated with a disease or condition, as set forth herein, for either therapeutic or diagnostic purposes. In one embodiment, a general range of virus administration is about 10³ to about 10⁷ plaque forming units (PFU) or more of virus per human subject, including about 10⁴ to about 10⁵ PFU virus per human subject.

Administration of a composition that elicits an immune response to reduce or prevent a mumps virus infection, can, but does not necessarily completely, eliminate such an infection, so long as the infection is measurably diminished. For example, administration of an effective amount of the composition can decrease the mumps virus infection (for example, as measured by infection of cells, or by number or percentage of subjects infected by mumps virus) by a desired amount, for example by at least 10%, at least 20%, at least 50%, at least 60%, at least 70%, at least 80%, at least 90%, at least 95%, at least 98%, or even 100% (elimination or prevention of detectable mumps virus infection, as compared to a suitable control).

In some embodiments, administration of a therapeutically effective amount of one or more of the disclosed recombinant mumps viruses to a subject induces a neutralizing immune response in the subject. To assess neutralization activity, following immunization of a subject, serum can be collected from the subject at appropriate time points, frozen, and stored for neutralization testing. Methods to assay for neutralization activity are known to the person of ordinary skill in the art, and include, but are not limited to, plaque reduction neutralization (PRNT) assays, microneutralization assays, flow cytometry based assays, and single-cycle infection assays.

In certain embodiments, the recombinant mumps virus can be administered sequentially with other anti-mumps virus therapeutic agents, such as before or after the other agent. Sequential administration can mean immediately following or after an appropriate period of time, such as hours, days, weeks, months, or even years later.

The following examples are provided to illustrate certain particular features and/or embodiments. These examples should not be construed to limit the disclosure to the particular features or embodiments described.

### EXAMPLES

### Example 1: Generation of RecombiMumps-G(dV), an Attenuated Mumps Virus Vaccine Expressing the F and HN Proteins of Genotype G Strains

RecombiMumps-G(dV) is a full-length infectious mumps virus clone rescued from a cDNA plasmid containing the complete nucleotide sequence of the Genotype A Jeryl Lynn (JL) strain of mumps virus to which:

(a) the JL fusion (F) and hemagglutinin-neuraminidase (HN) genes were removed and replaced with F and HN gene sequences synthesized based on a consensus sequence among all Genotype G F and HN sequences in GenBank, and

(b) the V gene was edited to include three stop codons to prevent a V protein from being translated from the V gene mRNA.

The plasmid encoding RecombiMumps-G(dV) (pJL-G(dV)) was rescued on BHK-BSR-T7 cells using helper plasmids expressing the nucleoprotein (N), phosphoprotein (P), and large protein (L) genes of the JL mumps virus strain. After rescue, the virus was expanded on Vero cells by a single passage to produce the Master Virus Seed Stock.

### Phase I: Production of Plasmid pJL

Plasmid pJL (a plasmid encoding the full length JL virus) was synthesized from multiple cDNA fragments including sequences encoding regulatory elements needed for cloning and rescue and sequences encompassing the entire JL strain. The regulatory elements used and the general cloning strategy employed was modeled after that published by Clarke et al., J Virol 74:4831-4838, (2000) and Sidhu et al., Virology 208:800-807 (1995). In total, 7 cDNA fragments were used. These were successively ligated into the multiple cloning site of the pBluescript II SK (+/−) vector (hereinafter “pBluescript”) purchased from Agilent Technologies Inc. (Santa Clara, Calif.).

The JL sequences used were based on the GENBANK™ Accession No. FJ211586. The 7 fragments designed for this effort are described below and were submitted to GenScript Corporation (Piscataway, N.J.) for synthesis.

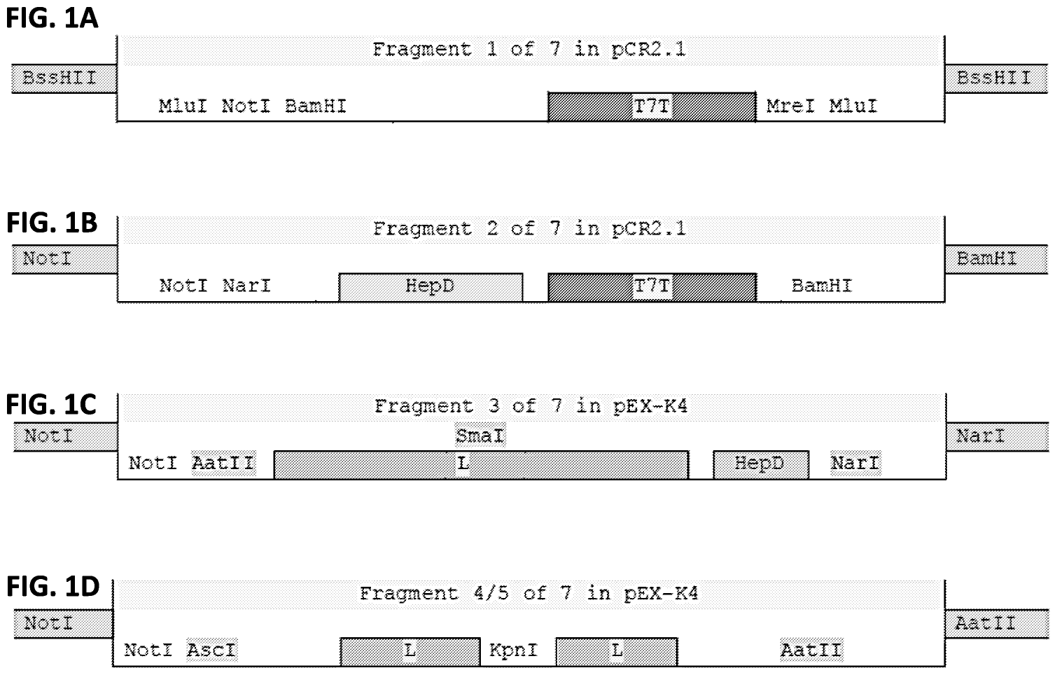

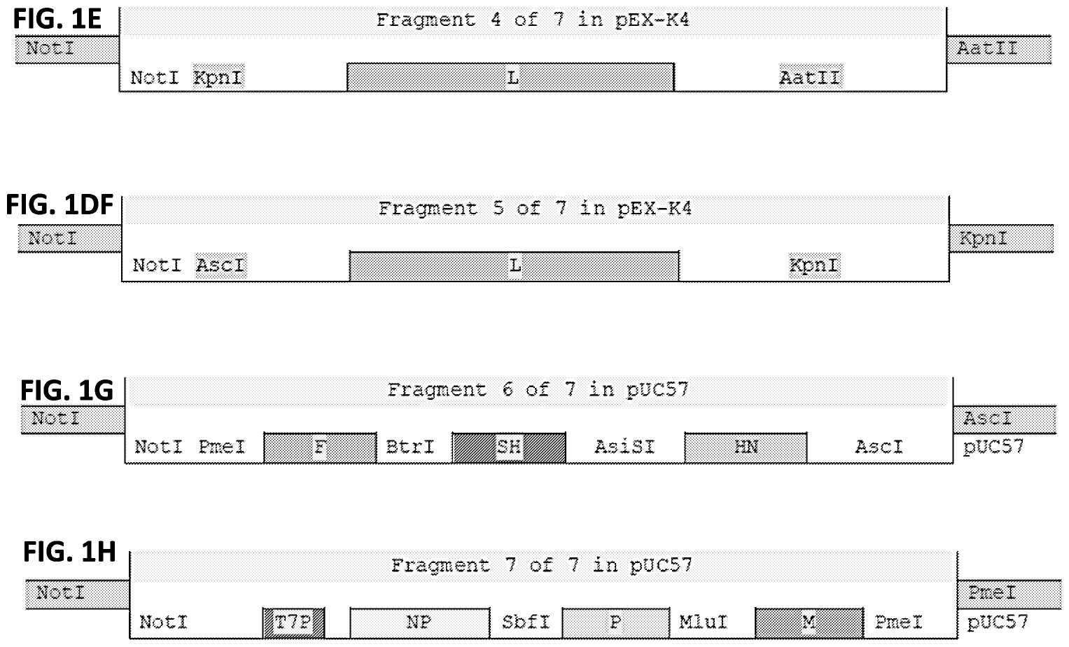

Fragment 1 of 7 contained a T7 termination sequence and restriction sites needed for future cloning steps, as shown in FIG. 1A. pBluescript vector BssHII restriction sites are indicated.

This fragment was obtained from GenScript in a pCR2.1 plasmid, which upon receipt was digested with restriction enzyme MluI. The resulting 175 base pair (bp) insert was gel purified and ligated into the BssHII digested, dephosphorylated (alkaline phosphatase, Roche Diagnostics Corp, Indianapolis, Ind.) and gel purified pBluescript vector. After ligation, the modified pBluescript vector was transformed into JM109 bacterial cells and plated on Lysogeny broth (LB) plates containing ampicillin. Plated colonies were picked and amplified overnight in LB containing ampicillin. Plasmid DNA was prepared using the QIAprep Spin Miniprep Kit (Qiagen, Gaithersburg, Md.). The pBluescript vector now containing Fragment 1 was digested with NotI and BamHI to insert Fragment 2 which contained a second T7 termination sequence, a portion of the hepatitis delta ribozyme sequence, and restriction sites needed for future cloning steps (FIG. 1B).

The pCR2.1 plasmid containing Fragment 2 was digested with NotI and BamHI and the resulting 222 bp fragment was gel purified and ligated into the NotI/BamHI digested pBluescript vector containing Fragment 1. This was transformed into bacteria, amplified, and purified as above. The pBluescript vector now containing Fragments 1 and 2 was digested with NarI, gel purified and then digested with NotI, dephosphorylated with calf intestinal phosphatase (CIP, New England BioLabs Inc., Ipswich, Mass.) and column purified to prepare it for incorporation of Fragment 3 which contained the rest of the hepatitis delta ribozyme sequence, part of the mumps virus L gene sequence, and restriction sites needed for future cloning steps (FIG. 1C).

Fragment 3 was provided in a pEX-K4 plasmid, which was digested with NarI, column purified and then digested with NotI. The resulting 3510 bp insert was gel purified and ligated with the NarI/NotI digested pBluescript vector containing Fragments 1 and 2. This was transformed into bacteria, amplified, and purified as above. The pBluescript vector now containing Fragments 1, 2, and 3 was digested with NotI, column purified, digested with AatII, column purified, dephosphorylated with CIP and column purified to prepare it for incorporation of Fragment 4/5, which contained the remaining portions of the L gene and restriction sites needed for future cloning steps (FIG. 1D).

Fragment 4/5 is the product of ligating Fragment 4 (FIG. 1E) into Fragment 5 (FIG. 1F). These needed to be synthesized separately due to the presence of an inverted repeat. Briefly, the pEX-K4 plasmid containing Fragment 4 above was digested with KpnI, column purified and then digested with NotI, column purified, dephosphorylated with CIP and column purified. Next, the pEX-K4 plasmid containing Fragment 5 was digested with KpnI, column purified and then digested with NotI and PstI. The resulting 2095 bp mumps fragment was gel purified and then ligated with the KpnI/NotI digested pEX-K4 plasmid containing Fragment 4. This was transformed into bacteria, amplified, and purified as above. The resulting plasmid containing Fragments 4/5 was digested with AatII, column purified and then digested with NotI. The resulting 3544 bp insert was gel purified and then ligated with the NotI/AatII digested pBluescript vector containing Fragments 1, 2 and 3. This was transformed into bacteria, amplified, and purified as above. The pBluescript vector now containing Fragments 1, 2, 3, 4 and 5 was digested with NotI, column purified, digested with AscI, dephosphorylated with CIP and column purified to prepare it for incorporation of Fragment 6, which contained the mumps virus F, SH and HN genes and restriction sites needed for future cloning steps (FIG. 1G).

Fragment 6 was provided in a pUC57 plasmid, which was digested with AscI, column purified and then digested with NotI. The resulting 3950 bp insert was gel purified and then ligated with the NotI/AscI digested pBluescript vector containing Fragments 1, 2, 3, 4 and 5. This was transformed into bacteria, amplified, and purified as above. The pBluescript vector now containing Fragments 1, 2, 3, 4, 5 and 6 was digested with PmeI, then NotI and CIP dephosphorylated to prepare it for incorporation of Fragment 7, which contained the truncated T7 promoter, and the mumps virus N, P, and M genes (FIG. 1H).

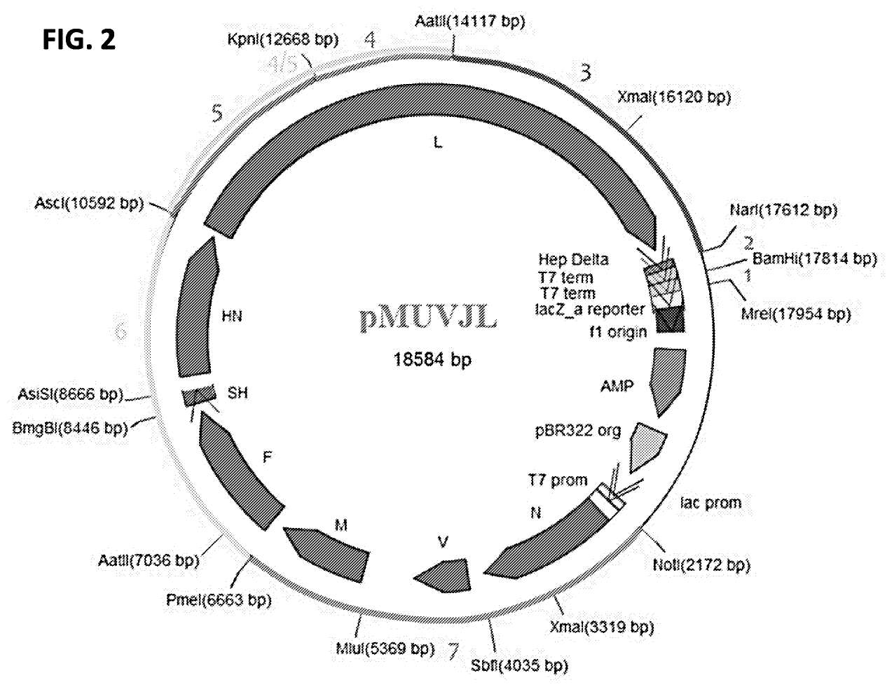

The pUC57 plasmid containing Fragment 7 was digested with PmeI and then NotI. The resulting 4491 bp insert was gel purified and then ligated with the PmeI/NotI digested pBluescript vector containing Fragments 1, 2, 3, 4, 5, and 6. This was transformed into bacteria, amplified, and purified as above. The pBluescript vector now contained all 7 JL fragments. A vector map showing the location of these 7 successively ligated fragments is shown in FIG. 2. This plasmid was termed “pJL.”

Phase II: Replacement of the JL F and HN Genes in pJL with Those of Genotype G Origin:

GenBank was queried for all mumps virus F and HN complete gene sequences from Genotype G strain viruses. In total, 120 F gene sequences and 214 HN genes sequences were identified. These were aligned and consensus F and HN sequences were determined. These sequences are shown below.

Consensus of Mumps genotype G, F gene
(SEQ ID NO: 2)
ATGAAGGTTTCTTTAGTTACTTGCTTGGGCTTTGCAGTCTTTTCATTTTC
CATATGTGTGAATATCAATATCTTGCAGCAAATTGGATATATCAAGCAAC
AAGTCAGGCAACTGAGCTATTACTCACAAAGTTCAAGCTCCTACATAGTG
GTCAAGCTTTTACCGAATATCCAACCCACTGATAACAGCTGTGAATTCAA
GAGTGTAACTCAATACAATAAGACCTTGAGTAATTTGCTTCTTCCAATTG
CAGAAAACATAAATAATATTGCATCGCCCTCACCTGGATCAAGACGTCAT
AAAAGGTTTGCTGGCATTGCCATTGGCATTGCTGCACTCGGTGTTGCAAC
CGCAGCACAAGTAACCGCCGCTGTCTCATTAGTTCAAGCACAGACAAATG
CACGCGCAATAGCGGCGATGAAAAATTCAATACAGGCAACTAATCGAGCA
GTCTTCGAAGTGAAAGAAGGCACCCAACAGTTAGCTATAGCGGTACAAGC
AATACAGAACCACATCAATACTATTATGAACACCCAATTGAACAATATGT
CCTGTCAGATTCTTGATAACCAGCTTGCAACCTCCCTGGGATTATACCTA
ACAGAATTAACAACAGTGTTTCAGCCACAATTAATTAATCCGGCATTGTC
ACCGATTAGTATACAAGCCTTGAGGTCTTTGCTTGGAAGTATGACACCTG
CAGTGGTTCAAGCAACATTATCTACTTCAATTTCTGCTGCTGAAATACTA
AGTGCCGGTCTAATGGAGGGTCAGATTGTTTCTGTTCTGCTGGATGAGAT
GCAGATGATAGTTAAGATAAATATTCCAACCATTGTCACACAATCAAATG
CATTGGTGATTGACTTCTACTCAATTTCGAGCTTTATTAATAATCAGGAA
TCCATAATTCAATTACCAGACAGGATCTTGGAGATCGGGAATGAACAATG
GAGCTATCCAGCAAAAAATTGTAAGTTGACAAGACACAACATATTCTGCC
AATACAATGAGGCAGAGAGGCTGAGCTTAGAATCAAAACTATGCCTTGCA
GGCAATATAAGTGCCTGTGTGTTCTCACCCATAGCAGGGAGTTATATGAG
GCGATTTGTAGCACTGGATGGAACAATTGTTGCAAACTGTCGAAGTCTAA
CGTGTCTATGCAAGAGTCCATCTTATCCTATATACCAACCTGACCATCAT
GCAGTCACGACCATTGATCTAACCGCATGTCAGACGTTGTCCCTAGACGG
ATTGGATTTCAGCATTGTCTCTCTAAGCAACATCACTTACGCTGAGAACC
TTACCATTTCATTGTCTCAGACAATCAATACTCAACCCATTGACATATCA
ACTGAACTGATCAAGGTCAATGCATCCCTCCAAAATGCCGTTAAGTACAT
AAAGGAGAGCAACCATCAACTCCAATCTGTGAGTATAAATTCTAAAATCG
GAGCTATAATCATAGCAGCCTTAGtTTTGAGCATCCTGTCAATGATCATT
TCACTGTTGTTTTGCTGCTGGGCTTACATTGCAACTAAAGAGATCAGAAG
AATCAACTTCAAAACAAATCATATCAACACAATATCAAGTAGTGTCGATG
ATCTCATCAGGTACTAA
Consensus of Mumps genotype G, HN gene
(SEQ ID NO: 3)
ATGGAGCCCTCGAAATTCTTCACAATATCGGACAGTGCCACCTTTGCACC
TGGGCCTGTTAGCAATGCGGCTAACAAGAAGACATTCCGAACCTGCTTCC
GAATACTGGCACTATCTGTACAAGCTGTCACCCTTATATTAGTTATTGTC
ACTTTAGGTGAGCTTGTAAGGATGATCAATGATCAAGGCTTGAGCAATCA
GTTGTCTTCAATTACAGACAAGATAAGAGAGTCAGCTACTATGATTGCAT
CTGCTGTGGGAGTAATGAATCAAGTTATTCATGGAGTAACGGTATCCTTA
CCCCTACAAATTGAGGGAAACCAAAATCAATTGTTAGCCACACTTGCCAC
AATCTGCACCAGCCAAAAACAAGTCTCAAACTGCTCTACAAACATCCCCT
TAGTCAATGACCTCAGGTTTATAAATGGGATCAATAAATTCATCATTGAA
GATTACGCAACTCATGATTTCTCTATCGGCCATCCACTCAATATGCCCAG
CTTTATCCCAACTGCAACTTCACCCAATGGTTGCACAAGAATTCCATCCT
TTTCTTTAGGTAAGACACACTGGTGCTACACACATAATGTAATTAATGCC
AACTGCAAGGACCATACTTCGTCTAACCAATATGTGTCCATGGGGATTCT
CGTTCAGACCGCGTCAGGTTATCCTATGTTCAAAACCTTAAAAATCCAAT
ATCTCAGTGATGGCCTGAATCGGAAAAGCTGCTCAATTGCAACAGTCCCT
GATGGGTGCGCGATGTACTGTTATGTCTCAACTCAACTTGAAACCGACGA
CTATGCGGGGTCCAGTCCACCCACCCAAAAACTTACCCTGTTATTCTATA
ATGACACCGTCACAGAAAGGACAATATCTCCATCTGGTCTTGAAGGGAAT
TGGGCTACTTTGGTGCCAGGAGTGGGGAGTGGGATATATTTTGAGAATAA
GTTGATCTTCCCTGCATATGGTGGTGTCTTGCCCAATAGTACACTCGGGG
TTAAATCAGCAAGAGAATTTTTCCGGCCTGTTAATCCATATAATCCATGT
TCAGGACCACAACAAGATTTAGACCAGCGTGCTTTGAGGTCATACTTCCC
AAGTTATTTCTCTAATCGAAGAATACAGAGTGCATTTCTTGTCTGTGCCT
GGAATCAGATCCTAGTTACAAATTGTGAGCTAGTTGTCCCCTCAAGCAAT
CAGACAATGATGGGTGCAGAAGGGAGAGTTTTATTGATCAATAATCGACT
ATTATATTATCAGAGAAGTACCAGCTGGTGGCCGTATGAACTCCTCTACG
AGATATCATTCACATTTACAAACTCTGGTCCATCATCTGTAAATATGTCC
TGGATACCTATATATTCATTCACTCGTCCTGGTTCAGGCAATTGCAGTGG
TGAAAATGTGTGCCCGACTGCTTGTGTGTCAGGGGTTTATCTTGATCCCT
GGCCATTAACTCCATATAGCCACCAATCAGGTATTAACAGAAATTTCTAT
TTCACAGGTGCTCTATTAAATTCAAGTACAACTAGAGTAAATCCTACCCT
TTATGTCTCTGCTCTTAATAATCTTAAAGTATTAGCCCCATATGGTACTC
AAGGACTGTTTGCCTCGTACACCACAACCACCTGCTTTCAAGATACCGGT
GATGCTAGTGTGTATTGTGTTTATATTATGGAACTAGCATCAAATATTGT
TGGAGAATTCCAAATTCTACCTGTGCTAACTAGATTGACTATCACTTGA

### Synthesis of the Consensus Genotype G, F Gene

The above consensus F gene was synthesized to include a 105 nucleotide sequence upstream of the ATG gene start sequence and a 329 nucleotide sequence downstream of the TAA stop sequence (both indicated by underline), provided in a pUC57 vector. The 105 nucleotide upstream sequence corresponds to the Jeryl Lynn sequence for that region modified to create a PmeI (GTTTAAAC; in bold) restriction site (substituted nucleotides shown in lowercase). The 329 nucleotide downstream sequence corresponds to the Jeryl Lynn sequence for that region containing the SH gene followed by mutations to create an AsiSI (GCGATCGC; in bold) site and MluI (ACGCGT; in bold) and SalI (GTCGAC; in bold) sites (substituted nucleotides shown in lowercase). The sequences for the latter two restriction sites were for intermediate cloning purposes only and are not incorporated into the final full-length vector.

(SEQ ID NO: 4)
GGGCGCGCCTGCAGGTAATTCGtTTaAAcTTATAGAAAAAATAAGCCTAG
AAGGATATCCTACTTCTCGACTTTCCAACTTTGAAAATAGAATAGATCAG
TAATCATGAAGGTTTCTTTAGTTACTTGCTTGGGCTTTGCAGTCTTTTCA
TTTTCCATATGTGTGAATATCAATATCTTGCAGCAAATTGGATATATCAA
GCAACAAGTCAGGCAACTGAGCTATTACTCACAAAGTTCAAGCTCCTACA
TAGTGGTCAAGCTTTTACCGAATATCCAACCCACTGATAACAGCTGTGAA
TTCAAGAGTGTAACTCAATACAATAAGACCTTGAGTAATTTGCTTCTTCC
AATTGCAGAAAACATAAATAATATTGCATCGCCCTCACCTGGATCAAGAC
GTCATAAAAGGTTTGCTGGCATTGCCATTGGCATTGCTGCACTCGGTGTT
GCAACCGCAGCACAAGTAACCGCCGCTGTCTCATTAGTTCAAGCACAGAC
AAATGCACGCGCAATAGCGGCGATGAAAAATTCAATACAGGCAACTAATC
GAGCAGTCTTCGAAGTGAAAGAAGGCACCCAACAGTTAGCTATAGCGGTA
CAAGCAATACAGAACCACATCAATACTATTATGAACACCCAATTGAACAA
TATGTCCTGTCAGATTCTTGATAACCAGCTTGCAACCTCCCTGGGATTAT
ACCTAACAGAATTAACAACAGTGTTTCAGCCACAATTAATTAATCCGGCA
TTGTCACCGATTAGTATACAAGCCTTGAGGTCTTTGCTTGGAAGTATGAC
ACCTGCAGTGGTTCAAGCAACATTATCTACTTCAATTTCTGCTGCTGAAA
TACTAAGTGCCGGTCTAATGGAGGGTCAGATTGTTTCTGTTCTGCTGGAT
GAGATGCAGATGATAGTTAAGATAAATATTCCAACCATTGTCACACAATC
AAATGCATTGGTGATTGACTTCTACTCAATTTCGAGCTTTATTAATAATC
AGGAATCCATAATTCAATTACCAGACAGGATCTTGGAGATCGGGAATGAA
CAATGGAGCTATCCAGCAAAAAATTGTAAGTTGACAAGACACAACATATT
CTGCCAATACAATGAGGCAGAGAGGCTGAGCTTAGAATCAAAACTATGCC
TTGCAGGCAATATAAGTGCCTGTGTGTTCTCACCCATAGCAGGGAGTTAT
ATGAGGCGATTTGTAGCACTGGATGGAACAATTGTTGCAAACTGTCGAAG
TCTAACGTGTCTATGCAAGAGTCCATCTTATCCTATATACCAACCTGACC
ATCATGCAGTCACGACCATTGATCTAACCGCATGTCAGACGTTGTCCCTA
GACGGATTGGATTTCAGCATTGTCTCTCTAAGCAACATCACTTACGCTGA
GAACCTTACCATTTCATTGTCTCAGACAATCAATACTCAACCCATTGACA
TATCAACTGAACTGATCAAGGTCAATGCATCCCTCCAAAATGCCGTTAAG
TACATAAAGGAGAGCAACCATCAACTCCAATCTGTGAGTATAAATTCTAA
AATCGGAGCTATAATCATAGCAGCCTTAGtTTTGAGCATCCTGTCAATGA
TCATTTCACTGTTGTTTTGCTGCTGGGCTTACATTGCAACTAAAGAGATC
AGAAGAATCAACTTCAAAACAAATCATATCAACACAATATCAAGTAGTGT
CGATGATCTCATCAGGTACTAATCTTAGATTGGTGATTCGTCCTGCAATT
TTAAAAGATTTAGAAAAAAACTAAAATAAGAATGAATCTCCTAGGGTCGT
AACGTCACGTCACCCTGCCGTCGCACTATGCCGGCAATCCAACCTCCCTT
ATACCTAACATTTCTAGTGCTAATCCTTCTCTATCTCATCATAACCCTGT
ATGTCTGGACTATATTGACTATTAACTATAAGACGGCGGTGCGATATGCA
GCACTGTACCAGCGATCCTTCTCTCGCTGGGGTTTTGATCACTCACTCTA
GAAAGATCCCCAATTAGGACAAGTCgCGATCgcTCACGCTAcgcgtcGac
G

### Synthesis of the Consensus Genotype G, HN Gene

The above consensus HN gene was synthesized to include a 162 nucleotide sequence upstream of the ATG gene start sequence and a 56 nucleotide sequence downstream of the TGA stop sequence (both sequences are underlined below), provided in a pEX-K248 vector. The 162 nucleotide upstream sequence corresponds to the Jeryl Lynn sequence for that region modified to create NotI (GCGGCCGC) and AsiSI (GCGATCGC) restriction sites (both sites shown in bold; nucleotide substitutions shown in lowercase). The 56 nucleotide downstream sequence corresponds to the Jeryl Lynn sequence for that region modified to create AscI (GGCGCGCC), KpnI (GGTACC) and XhoI (CTCGAG) restriction sites (all three sites shown in bold; nucleotide substitutions shown in lowercase).

(SEQ ID NO: 5)
gcggccgCCAAGTCgCGATCgcTCACGCTAGAACAAGCTGCATTCAAATG
AAGCTGTGCTACCATGAGACATAAAGAAAAAAGCAAGCCAGAACAAACCT
AGGATCATAACACAATACAGAATATTAGCTGCTATCACAACTGTGTTCCG
GCCACTAAGAAAATGGAGCCCTCGAAATTCTTCACAATATCGGACAGTGC
CACCTTTGCACCTGGGCCTGTTAGCAATGCGGCTAACAAGAAGACATTCC
GAACCTGCTTCCGAATACTGGCACTATCTGTACAAGCTGTCACCCTTATA
TTAGTTATTGTCACTTTAGGTGAGCTTGTAAGGATGATCAATGATCAAGG
CTTGAGCAATCAGTTGTCTTCAATTACAGACAAGATAAGAGAGTCAGCTA
CTATGATTGCATCTGCTGTGGGAGTAATGAATCAAGTTATTCATGGAGTA
ACGGTATCCTTACCCCTACAAATTGAGGGAAACCAAAATCAATTGTTAGC
CACACTTGCCACAATCTGCACCAGCCAAAAACAAGTCTCAAACTGCTCTA
CAAACATCCCCTTAGTCAATGACCTCAGGTTTATAAATGGGATCAATAAA
TTCATCATTGAAGATTACGCAACTCATGATTTCTCTATCGGCCATCCACT
CAATATGCCCAGCTTTATCCCAACTGCAACTTCACCCAATGGTTGCACAA
GAATTCCATCCTTTTCTTTAGGTAAGACACACTGGTGCTACACACATAAT
GTAATTAATGCCAACTGCAAGGACCATACTTCGTCTAACCAATATGTGTC
CATGGGGATTCTCGTTCAGACCGCGTCAGGTTATCCTATGTTCAAAACCT
TAAAAATCCAATATCTCAGTGATGGCCTGAATCGGAAAAGCTGCTCAATT
GCAACAGTCCCTGATGGGTGCGCGATGTACTGTTATGTCTCAACTCAACT
TGAAACCGACGACTATGCGGGGTCCAGTCCACCCACCCAAAAACTTACCC
TGTTATTCTATAATGACACCGTCACAGAAAGGACAATATCTCCATCTGGT
CTTGAAGGGAATTGGGCTACTTTGGTGCCAGGAGTGGGGAGTGGGATATA
TTTTGAGAATAAGTTGATCTTCCCTGCATATGGTGGTGTCTTGCCCAATA
GTACACTCGGGGTTAAATCAGCAAGAGAATTTTTCCGGCCTGTTAATCCA
TATAATCCATGTTCAGGACCACAACAAGATTTAGACCAGCGTGCTTTGAG
GTCATACTTCCCAAGTTATTTCTCTAATCGAAGAATACAGAGTGCATTTC
TTGTCTGTGCCTGGAATCAGATCCTAGTTACAAATTGTGAGCTAGTTGTC
CCCTCAAGCAATCAGACAATGATGGGTGCAGAAGGGAGAGTTTTATTGAT
CAATAATCGACTATTATATTATCAGAGAAGTACCAGCTGGTGGCCGTATG
AACTCCTCTACGAGATATCATTCACATTTACAAACTCTGGTCCATCATCT
GTAAATATGTCCTGGATACCTATATATTCATTCACTCGTCCTGGTTCAGG
CAATTGCAGTGGTGAAAATGTGTGCCCGACTGCTTGTGTGTCAGGGGTTT
ATCTTGATCCCTGGCCATTAACTCCATATAGCCACCAATCAGGTATTAAC
AGAAATTTCTATTTCACAGGTGCTCTATTAAATTCAAGTACAACTAGAGT
AAATCCTACCCTTTATGTCTCTGCTCTTAATAATCTTAAAGTATTAGCCC
CATATGGTACTCAAGGACTGTTTGCCTCGTACACCACAACCACCTGCTTT
CAAGATACCGGTGATGCTAGTGTGTATTGTGTTTATATTATGGAACTAGC
ATCAAATATTGTTGGAGAATTCCAAATTCTACCTGTGCTAACTAGATTGA
CTATCACTTGAGTTGTAGTGAATGTAGCAGGAAGCTTTACGGGCGcGcCT
CATTTCggtacctcgag

Incorporating the Consensus Genotype G, F and HN Genes into the pJL

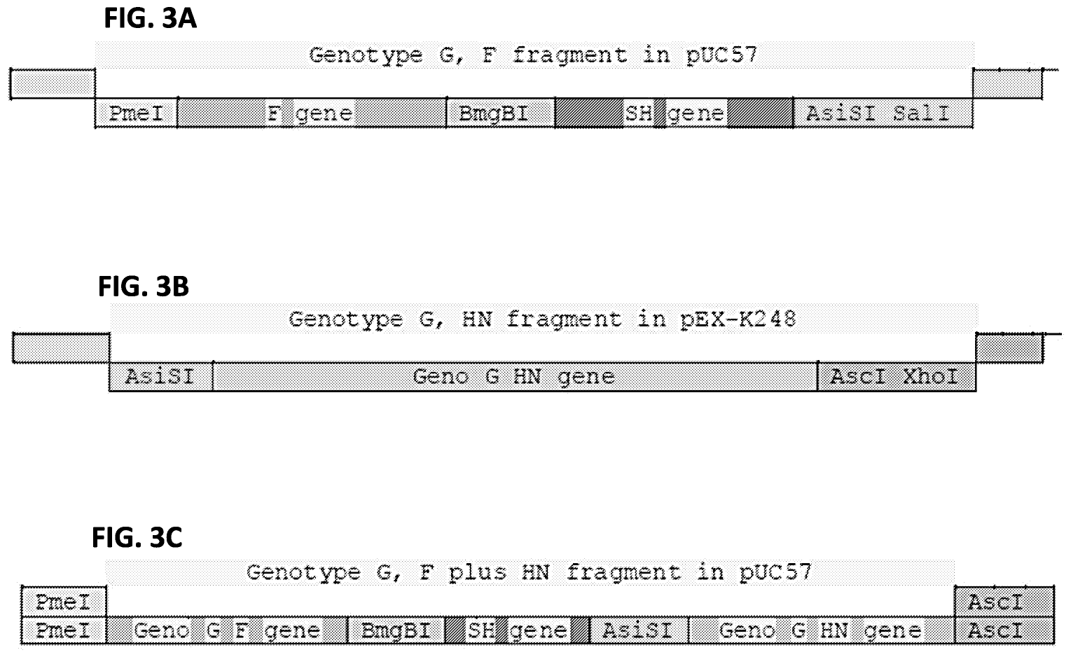

The above pUC57 plasmid containing the Genotype G, F and SH genes was digested with AsiSI and SalI and the resulting 4741 bp fragment was column purified (FIG. 3A).

The pEX-K248 plasmid containing the Genotype G, HN gene was digested with AsiSI and XhoI (compatible with the SalI site in the fragment) and the resulting 1943 bp HN gene fragment was gel purified and ligated into the AsiSI/SalI digested pUC57 plasmid (containing the F and SH genes), transformed into bacteria, amplified, and miniprep DNA was prepared. The XhoI and SalI sites were used for ligation but both sites are lost after ligation (FIG. 3B).

The pUC57 plasmid containing the genotype G, F and HN genes was digested with PmeI, AscI and ScaI, the 3929 bp genotype G, F/HN gene fragment was gel purified (FIG. 3C). The ScaI site is within the pUC57 plasmid and was used to differentiate between the PmeI/AscI fragment containing the F, SH and HN genes from the rest vector (which would be almost identical in size to the F/SH/HN containing fragment in the absence of cutting with SacI).

The pBluescript vector containing the Jeryl Lynn infectious cDNA was digested with PmeI and AscI, dephosphorylated and the 14655 bp vector fragment now minus the F, SH and HN genes was gel purified and then ligated with the PmeI/AscI genotype G, F/HN gene fragment. This was transformed into bacteria, amplified, and miniprep DNA was prepared. This plasmid DNA was checked by restriction endonuclease digests and sequenced to confirm the presence of the correct insert. After cloning this fragment into pJL, the resulting material was termed “pJL-G.”

Phase III: Creating the V Gene Stop Mutations in pJL-G to Produce pJL-G(dV)

pJL-G was modified to encode three translation stop sequences at the start of the unique portion of the V gene. The V gene encodes the V protein and the P and I proteins. Consequently, the first third of the V gene is shared among all three proteins, thus the translation stop signals could not be inserted into the shared region. Instead, they were introduced into the portion of the V-gene that is unique to the V protein.

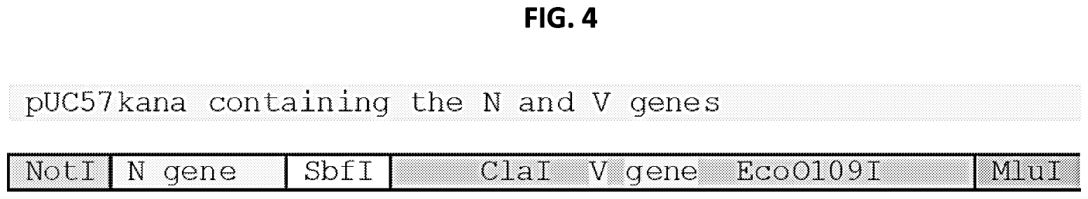

Incorporating the three V protein translation stop sequences into pJL-G was accomplished in a stepwise manner. First, a pUC57-Kan vector (GenScript) containing an unrelated insert was digested with NotI and MluI to remove the irrelevant sequence. The empty vector was then dephosphorylated with CIP and gel purified to prepare it for incorporation of a pJL-G DNA fragment that contains the V gene region of interest. For this, the entire pJL-G N and V genes were inserted. To do this, plasmid pJL-G was digested with NotI and MluI which flank the 3197 bp N/V gene. This piece was gel purified and ligated into the empty pUC57-Kan vector. Following transformation into bacteria, the plasmid was amplified and miniprep DNA was prepared. This plasmid is shown in FIG. 4.

The V gene contains a natural ClaI (ATCGAT; in bold) restriction site at genome position 2388 and a natural EcoO109I (GGGTCCC; in bold) restriction site at genome position 2830, flanking the region of interest where the novel translation stop codons were to be created. These restriction sites are in bold and the three new translation stop sequences are underlined in the sequence below (SEQ ID NO: 6; mutations in lowercase):

2388
ATCGATTTGTTGAGAAACCTAGAACCTCAACGCCGGTGACAGAATTTAAG
AGGGGGGCCGGGAGCGGCTGCTaAtGGCCAGACAATCCAAGAGGAGGGCA
TAGACGGtAATGGAGCCTCAGCTGGGTCtAAGGAGAGGTCCGGGTCTTTG
AGTGGTGCAACCCTATATGCTCACCTATCACTGCCGCAGCAAGATTCCAC
TCCTGCAAATGTGGGAATTGCCCCGCAAAGTGCGATCAGTGCGAACGAGA
TTATGGACCTCCTTAGGGGGATGGATGCTCGCCTGCAACATCTTGAACAA
AAGGTGGACAAGGTGCTTGCACAGGGCAGCATGGTGACCCAAATAAAGAA
TGAATTATCAACAGTAAAGACAACATTAGCAACAATTGAAGGGATGATGG
CAACAGTAAAGATCATGGATCCTGGAAATCCGACAGGGGTCCC 2830

This 443 bp sequence was synthesized (GenScript) and obtained in a pUC57 vector. The first translation stop codon (TAA) was the closest that could be engineered into the P gene unique region with minimal effect on the P protein amino acid sequence. Introduction of this “A” mutation would have resulted in an AAA codon in the V protein gene reading frame, resulting in a glutamine to lysine non-conservative amino acid change. Thus, the next nucleotide (T) was also mutated to change the codon to AAT to encode a more conservative amino acid change (asparagine). The other two translation stop codons do not alter the amino acid sequence of the P protein.

This pUC57-Kan plasmid was digested with ClaI and EcoO109I to remove the 443 bp stretch out of the N/V DNA fragment of pJL-G origin. In parallel, the pUC57 plasmid containing the mutated version of this region encoding the new translation stop codons was digested with the same restriction enzymes. The excised fragment with the stop codons was then gel purified and ligated into the digested pUC57-Kan vector containing pJL-G N and V sequences lacking the 443 bp region. This was transformed into bacteria, amplified and miniprep DNA was prepared. This plasmid DNA was checked by restriction endonuclease digests to confirm the presence of the correct insert. This plasmid, now containing N and V sequences with stop signals, was digested with NotI, MluI and PvuI and the 3197 bp modified fragment was gel purified to ready it for insertion into pJL-G, which was digested with NotI and MluI, dephosphorylated with CIP, gel purified and then ligated with the 3197 bp N and V-stop fragment. This was transformed into bacteria, amplified and miniprep DNA was prepared. This plasmid DNA was checked by restriction endonuclease digests and sequenced to confirm the presence of the correct insert. This pJL-G vector now containing the V gene translation stop signals was termed “pJL-G(dV).”

Phase IV: Rescue of RecombiMumps-G(dV) from Plasmid pJL-G(dV)

### Construction of the Mumps Virus Helper Clones

The mumps virus N, P and L proteins are required to enable rescue of recombinant mumps viruses. Plasmids encoding these proteins must be co-transfected into cells along with the full-length mumps virus cDNA (pJL-G(dV)). Therefore, three helper plasmids needed to be generated, one expressing the N gene, one expressing the P gene, and one expressing the L gene. These are identified as plasmids pTM1-N, pTM1-P, and pTM1-L, respectively, and their construction is described below.

The first step was to modify the T7 promoter-driven expression vector “pTM1” (Moss et al., Nature 348:91-92, 1990). The two thymidine kinase regions (tk-L and tk-R) in pTM1 had to be removed to enable the restriction strategy for construction of the mumps helper clones. To do this, the plasmid was digested with XbaI and ClaI to remove the first tk region, phenol-chloroform purified and the overhanging ends filled in using the Klenow fragment of DNA polymerase. The end filled vector was self-ligated and transformed into bacteria, amplified, and purified. The resultant plasmid was termed “pTM1-tk”. This was then digested with BspEI and NaeI to remove the second tk region, phenol-chloroform purified and the overhanging ends filled in using the Klenow fragment of DNA polymerase. The end filled vector was self-ligated and transformed into bacteria, amplified, and purified. The resultant plasmid was termed “pTM1-2tk.”

Construction of Helper Clone pTM1-N

The mumps N sequence with overhangs containing restriction sites for cloning (1737 bp) is shown below. The sequence was submitted to GenScript for synthesis and was received in the pEX-K4 vector. Sequences in bold represent the NcoI and XhoI restriction sites. The start and stop codons for the N ORF are underlined.

(SEQ ID NO: 7)
ggtcgacgcgttgacactCCATGGAAAATGTCATCTGTGCTCAAGGCATT
TGAGCGGTTCACGATAGAACAGGAACTTCAAGACAGGGGTGAGGAGGGTT
CAATTCCACCGGAGACTTTAAAGTCAGCAGTCAAAGTCTTCGTTATTAAC
ACACCCAATCCCACCACACGCTATCAGATGCTAAACTTTTGCTTAAGAAT
AATCTGCAGTCAAAATGCTAGGGCATCTCACAGGGTAGGTGCATTGATAA
CATTATTCTCACTTCCCTCAGCAGGCATGCAAAATCATATTAGATTAGCA
GATAGATCACCCGAAGCTCAGATAGAACGCTGTGAGATTGATGGTTTTGA
GCCTGGTACATATAGGCTGATTCCAAATGCACGCGCCAATCTTACTGCCA
ATGAAATTGCTGCCTATGCTTTGCTTGCAGATGACCTCCCTCCAACCATA
AATAATGGAACTCCTTACGTACATGCAGATGTTGAAGGACAGCCATGTGA
TGAGATTGAGCAGTTCCTGGATCGGTGTTACAGTGTACTAATCCAGGCTT
GGGTAATGGTCTGTAAATGTATGACAGCGTACGACCAACCTGCCGGGTCT
GCTGATCGGCGATTTGCGAAATACCAGCAGCAAGGTCGCCTTGAGGCAAG
ATACATGCTGCAACCGGAGGCCCAAAGGTTGATTCAAACTGCCATCAGGA
AAAGTCTTGTTGTTAGACAGTACCTTACCTTCGAACTCCAGTTGGCGAGA
CGGCAGGGATTGCTATCAAACAGATACTATGCAATGGTGGGTGACATCGG
AAAGTACATTGAGAATTCAGGCCTTACTGCCTTCTTTCTCACTCTCAAAT
ATGCACTAGGGACCAAATGGAGTCCTCTATCATTGGCTGCATTCACCGGT
GAACTCACCAAGCTCCGATCCTTGATGATGTTATATCGAGGTCTCGGAGA
ACAAGCCAGATACCTTGCTCTGTTAGAGGCTCCCCAAATAATGGACTTTG
CACCCGGGGGCTACCCATTGATATTCAGTTATGCTATGGGAGTCGGTACA
GTCCTAGATGTTCAAATGCGAAATTACACTTATGCACGACCTTTCCTAAA
CGGTTATTATTTCCAGATTGGGGTTGAGACCGCACGAAGACAACAAGGCA
CTGTTGACAACAGAGTAGCAGATGATCTGGGCCTGACTCCTGAGCAAAGA
ACTGAGGTCACTCAGCTTGTTGACAGGCTTGCAAGGGGAAGAGGTGCTGG
GATACCAGGTGGGCCTGTGAATCCTTTTGTTCCTCCGGTTCAACAGCAAC
AACCTGCTGCCGTATATGAGGACATTCCTGCATTGGAGGAATCAGATGAC
GATGGTGATGAAGATGGAGGCGCAGGATTCCAAAATGGAGTACAATTACC
AGCTGTAAGACAGGGAGGTCAAACTGACTTTAGAGCACAGCCTTTGCAAG
ATCCAATTCAAGCACAACTTTTCATGCCATTATATCCTCAAGTCAGCAAC
ATGCCAAATAATCAGAATCATCAGATCAATCGCATCGGGGGGCTGGAACA
CCAAGATTTATTACGATACAACGAGAATGGTGATTCCCAACAAGATGCAA
GGGGCGAACACGTAAACACTTTCCCAAACAATCCCAATCAAAACGCACAG
TTGCAAGTGGGAGACTGGGATGAGTAAATCACTGACATGATCAAACTAAC
CCCAATCGCAACActcgagggacaatacgcgtcgacg

pTMI-2tk was digested with NcoI and then XhoI, CIP dephosphorylated and column purified. pEX-K4 containing the mumps N sequence was digested with NcoI, XhoI and BglII to release a 1699 bp fragment. The N fragment was gel purified, ligated into the NcoI/XhoI digested pTMI-2tk and transformed into bacteria, amplified, and purified as above. The resultant plasmid was termed “pTM1-N.”

Construction of Helper Clone pTM1-P

The mumps virus P sequence with overhangs containing restriction sites for cloning (1281 bp) is shown below. The sequence was submitted to GenScript for synthesis and was received in the pEX-K4 vector. Sequences in bold represent the NcoI and XhoI restriction sites. The start and stop codons for the P ORF are underlined. The two lowercase “g” residues in bold represent the synthetic addition of two nucleotides at the editing site to produce the P ORF.

(SEQ ID NO: 8)
ggtcgacgcgtgggcaagCCATGGATCAATTTATAAAACAGGATGAGACC
GGTGATTTAATTGAGACAGGAATGAATGTTGCGAATCATTTCCTATCCAC
CCCAATTCAGGGAACCAATTCGCTGAGCAAGGCCTCAATCCTCCCTGGTG
TTGCACCTGTACTCATTGGCAATCCAGAGCAAAAGAACATTCAGCACCCT
ACCGCATCACATCAGGGATCCAAGACAAAGGGCAGAGGCTCAGGAGTCAG
GTCCATCATAGTCTCACCCTCCGAAGCAGGCAATGGAGGGACTCAGATTC
CTGAGCCCCTTTTTGCACAAACAGGACAGGGTGGTATAGTCACCACAGTT
TACCAGGATCCAACTATCCAACCAACAGGTTCATACCGAAGTGTGGAATT
GGCGAAGATCGGAAAAGAGAGAATGATTAATCGATTTGTTGAGAAACCTA
GAACCTCAACGCCGGTGACAGAATTTAAGAggGGGGGGCCGGGAGCGGCT
GCTCAAGGCCAGACAATCCAAGAGGAGGGCATAGACGGGAATGGAGCCTC
AGCTGGGTCCAAGGAGAGGTCCGGGTCTTTGAGTGGTGCAACCCTATATG
CTCACCTATCACTGCCGCAGCAAGATTCCACTCCTGCAAATGTGGGAATT
GCCCCGCAAAGTGCGATCAGTGCGAACGAGATTATGGACCTCCTTAGGGG
GATGGATGCTCGCCTGCAACATCTTGAACAAAAGGTGGACAAGGTGCTTG
CACAGGGCAGCATGGTGACCCAAATAAAGAATGAATTATCAACAGTAAAG
ACAACATTAGCAACAATTGAAGGGATGATGGCAACAGTAAAGATCATGGA
TCCTGGAAATCCGACAGGGGTCCCAGTTGATGAGCTTAGAAGAAGTTTTA
GTGATCACGTGACAATTGTTAGTGGACCAGGAGATGTGTCGTTCAGCTCC
AGTGAAAAACCCACACTGTATTTGGATGAGCTGGCGAGGCCCGTCTCCAA
GCCTCGTCCTGCAAAGCAGACAAAATCCCAACCAGTAAAGGATTTAGCAG
GACAGAAAGTGATGATTACCAAAATGATCACTGATTGTGTGGCTAATCCT
CAAATGAAGCAGGCGTTCGAGCAACGATTGGCAAAGGCCAGCACCGAGGA
TGCTCTGAACGATATCAAGAGAGACATCATACGAAGCGCCATATGAATTC
ACCAGGAGCACCAGACTCAAGGAAAAATCTATGAACTGAGAGCCACAATG
ATTCCCTCTCGAGtagagcgacgcgtcgacg

pTMI-2tk was digested with NcoI and then XhoI, CIP dephosphorylated and column purified. pEX-K4 containing the mumps P was digested with NcoI and XhoI to release a 1243 bp fragment. The P fragment was gel purified, ligated into the NcoI/XhoI digested pTMI-2tk and transformed into bacteria, amplified, and purified as above. The resultant plasmid was termed “pTM1-P.”

Construction of Helper Clone pTM1-L

The mumps L ORF was cloned sequentially into pTM1-2tk in three steps as described below. Sequence for the 3′ end (351 bp fragment) of L was submitted to GenScript for synthesis and was received in the pCR2.1 vector. The remaining two fragments were obtained by restriction digestion from plasmid pJL (see above).

pTMI-2tk was digested with NcoI and PstI, dephosphorylated with CIP and column purified. pCR2.1 containing the L 3′ end was digested with PciI and PstI to release a 351 bp fragment. This fragment was gel purified, ligated into the NcoI/PstI digested pTMI-2tk and transformed into bacteria, amplified, and purified as above. NcoI is compatible with PciI.

pTM1-2tk containing the L 3′ end was digested with AatII and NcoI, dephosphorylated with CIP and column purified. Plasmid pJL was digested with AatII and NcoI to release a 3050 bp fragment. This fragment, corresponding to the middle region of L, was gel purified, ligated into the AatII/NcoI digested pTMI-2tk containing the 3′ of L and transformed into bacteria, amplified, and purified as above.

pTM1-2tk containing the middle and 3′ of L was digested with AscI and AatII, dephosphorylated with CIP and column purified. Plasmid pJL was also digested with AscI and AatII to release a 3529 bp fragment. This fragment, corresponding to the 5′ region of L was gel purified, ligated into the AscI/AatII digested pTMI-2tk containing the middle and 3′ of L and transformed into bacteria, amplified, and purified as above. The resultant plasmid was termed “pTM1-L.” The complete sequence of the L ORF cloned into pTM1-2tk is set forth as SEQ ID NO: 9.

Rescue of pJL-G(dV)

To rescue virus RecombiMumps-G(dV) from the plasmid pJL-G(dV), T7 polymerase-expressing BHK-BSRT7/5 cells in 6-well plates were transfected with pJL-G(dV) in the presence of LIPOFECTAMINE™ 2000 (Invitrogen) and helper plasmids pTM1-N, pTM1-P, and pTM1-L, all of which are under control of a T7 polymerase promoter. Two days post-transfection, the cells were trypsinized and transferred to a 75 cm² flask containing DMEM supplemented with 10% (v/v) FBS and incubated until cytopathic effects were observed (day 5). To amplify the rescued virus, the cell supernatant was transferred to a confluent monolayer of Vero cells in a 75 cm² flask containing DMEM supplemented with 10% (v/v) FBS and incubated for 3 days. The supernatant was collected and clarified by centrifugation at 10,000×g for 10 minutes to remove cellular debris, aliquoted, labeled as “RecombiMumps-G(dV) Master Virus Stock” and stored at −70° C. Lot numbers and certificates of analysis were obtained from all buffers, media, and media supplements used in the generation of the master virus stock.

### Phase VI: Testing of RecombiMumps-G(dV)

### Freedom of Adventitious Agents:

Assurance that the Master Virus Stock was free from adventitious agents was provided by deep sequencing. Briefly, an aliquot of the stock was treated with 50,000 gel units of micrococcal nuclease (New England Bio-Labs) for 2 hours at 37° C. to digest non-particle associated nucleic acids. Total RNA and DNA was then extracted using QIAamp Viral RNA Mini Kit (Qiagen) and DNeasy Blood & Tissue Kit (Qiagen) respectively, according to the manufacturer's protocols. 0.5 g of total RNA or DNA was fragmented by focused-ultrasonicator (Covaris) to generate fragments of approximately 250-300 bp. To prepare the RNA and DNA libraries from the fragments, the NEBNext mRNA Library Prep Master Mix Set for Illumina (New England Bio-Labs) was used according to the manufacturer's protocol, resulting in the fragments being ligated to Illumina paired end (PE) adaptors. These were then amplified using 12 cycles of PCR with multiplex indexed primers and purified by magnetic beads (Agencourt AMPure PCR purification system, BeckmanCoulter). After analyzing the libraries for size and quality (BioAnalyzer, Agilent Technologies, Inc.), deep sequencing was performed using MiSeq (Illumina) producing 250 nucleotide paired-end reads. The raw sequencing reads were analyzed by the CensuScope algorithm (Shamsaddini et al., BMC Genomics 15:918, 2014) using custom software. Briefly, to check the viral composition of the sample, 1000-10,000 sequencing reads were aligned against the viral genome references composed from all viral genomes present in NCBI GenBank. Sixty-one percent of all reads aligned to the expected mumps virus sequences. The remaining reads aligned to short fragments with homology to various plant and animal viruses, none longer than 400 nucleotides. Nearly all were known “contaminants” of cell culture and sequencing reagents and short viral fragments integrated into the Vero cell genome, the cell substrate used for virus production. The only full-length virus sequence present was RecombiMumps-G(dV).

### Attenuation:

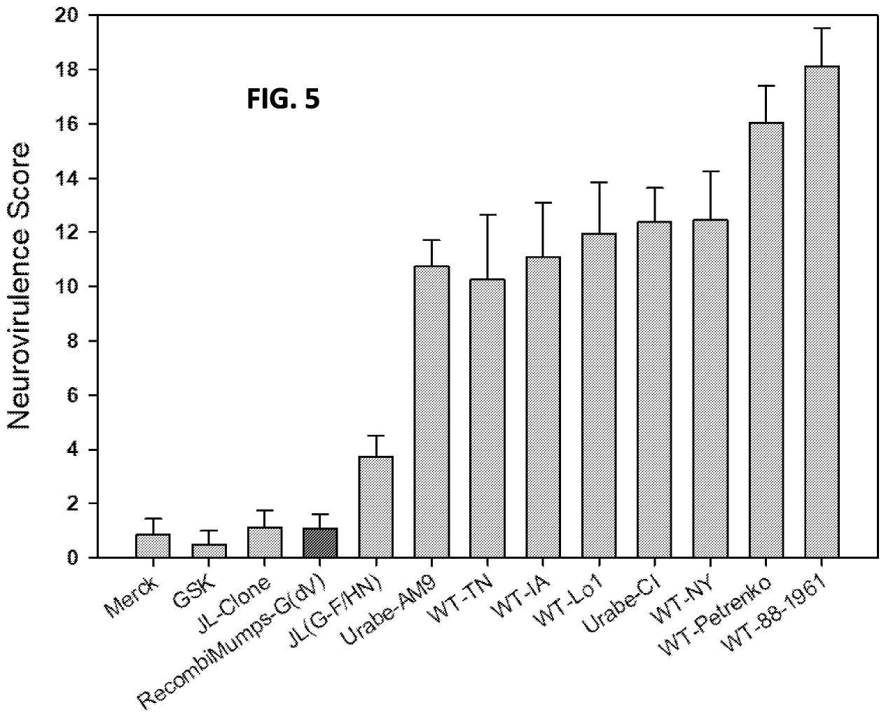

Pre-clinical safety testing of the Master Virus Stock was assessed in a rat model as described previously (Rubin et al., J Infect Dis 191(7):1123-1128, 2005). Briefly, newborn litters (<12 hours of age) from three pregnant Lewis rats (Envigo, Frederick, Md.) were inoculated intracerebrally with 100 plaque forming units (pfu) of the virus in a 20-μL volume of MEM using a 33-gauge needle and a Hamilton syringe in the left parietal area of the skull, ˜2 mm left of midline and midway between the bregma and lambda. A total of 26 rat pups were inoculated. On day 30 post-inoculation, brains were removed and divided sagittally at anatomical midline and fixed in 10% neutral buffered formalin. The fixed brain hemispheres were then paraffin embedded and one 8-10 μm thick sagittal section was taken at a standard distance from either side of the anatomical midline, adhered to a glass slide, and stained with hematoxylin and eosin. The stained brain sections were placed on a flat-bed scanner and uploaded to a computer as a jpeg file. The neurovirulence score was determined by measuring the cross-sectional pixel area of the brain (excluding the cerebellum) and that of the lateral ventricle on each tissue section using the open source image processing program ImageJ (NIH). The mean ratio (percentage) of these two measurements on each of the two tissue sections per rat brain is the neurovirulence score for that brain. The neurovirulence score for the virus inoculum was determined as the mean neurovirulence score for all brains. The neurovirulence score for RecombiMumps-G(dV) was 1.1, which is not statistically different from the neurovirulence score determined for the FDA-licensed Jeryl Lynn vaccine strain (0.9). For comparison, the neurovirulence score for the Urabe-AM9 vaccine strain which was discontinued due to reactogenicity was 10.7 and the neurovirulence scores for several wild type viruses ranged from 10.2 to 18.1, as shown in FIG. 5.

### Example 2: Immunogenicity of RecombiMumps-G(dV) in Non-Human Primates

This example describes an immunogenicity study of RecombiMumps-G(dV) in non-human primates.

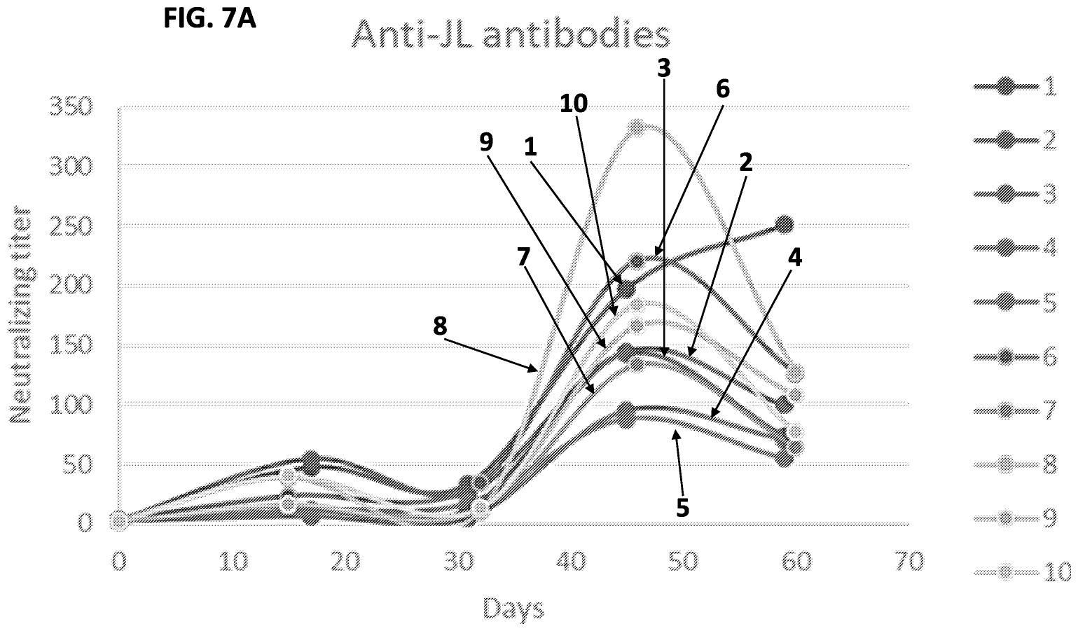

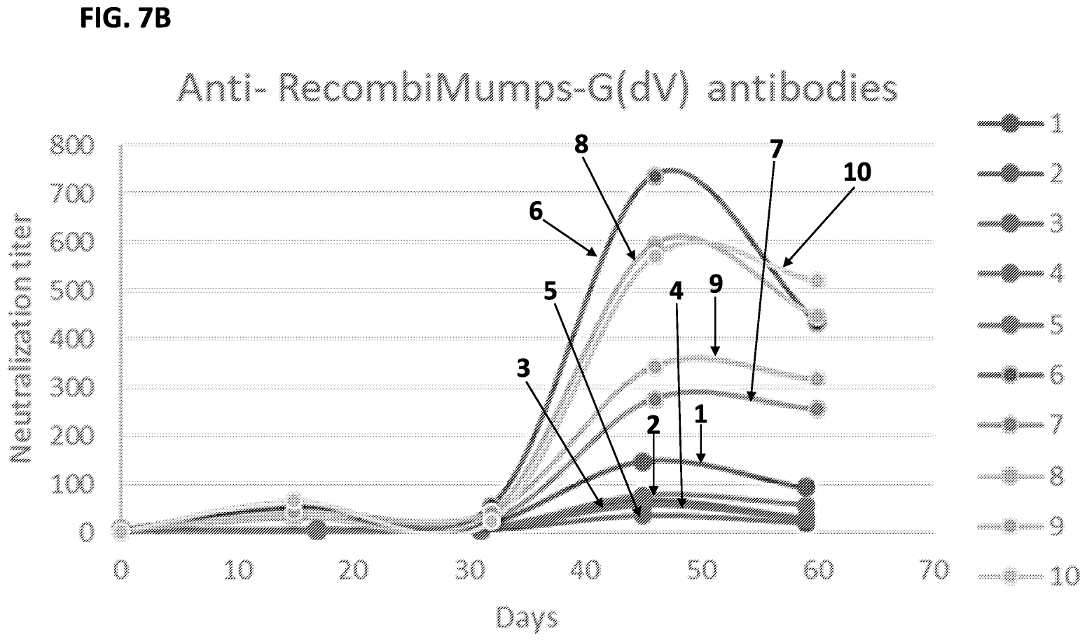

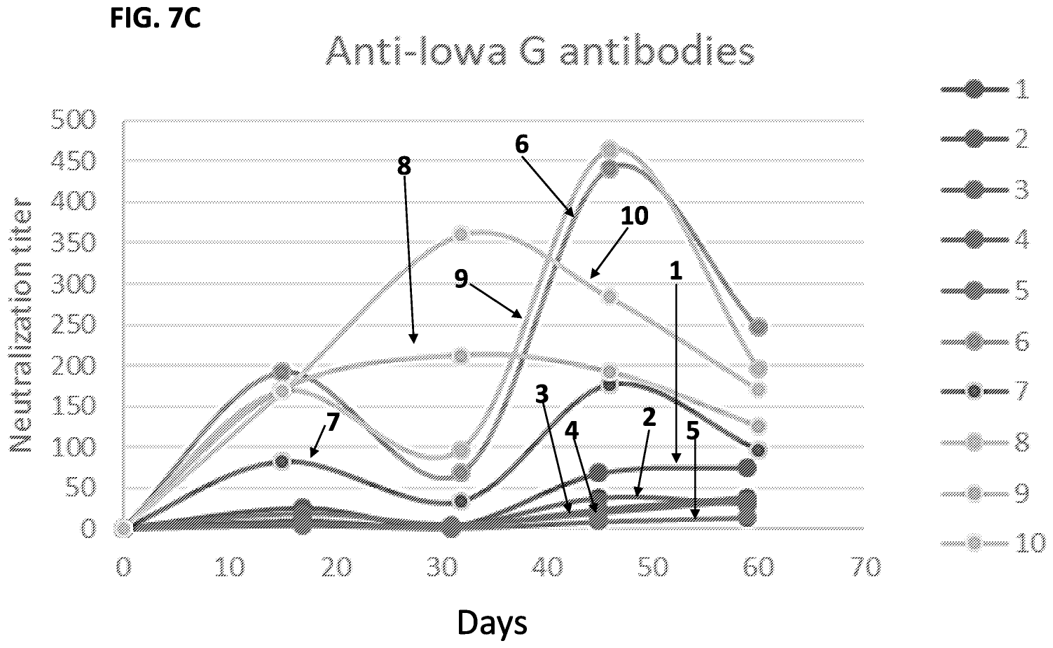

Monkeys (Rhesus macaque) were immunized intramuscularly with either the Jeryl Lynn vaccine virus strain (JL; produced as described in Example 1) or RecombiMumps-G(dV) and then boosted with a second dose 30 days later. Each dose consisted of 1.6×10⁵ pfu of virus in 0.4 mL buffer, similar to a human dose. Sera were collected before vaccination (day 0) and approximately on days 15, 30, 45 and 60. At each time point, the sera were tested for their ability to neutralize JL and RecombiMumps-G(dV). The results are shown in FIGS. 7A-7B. Lines 1-5 represent the five monkeys vaccinated with JL and lines 6-10 represent the five monkeys vaccinated with RecombiMumps-G(dV). The inflection point (day 30) represents administration of the second dose (booster dose).

As shown in FIGS. 7A-7B, titers from all 10 monkeys were similarly low against both viruses following the first dose (days 15 and 30). However, following the second dose, a clear boosting effect was observed with peak antibody titers occurring on day 45 (2 weeks after the second dose). At this timepoint, neutralizing antibody titers against JL (FIG. 7A) were more than 50% higher in animals receiving RecombiMumps-G(dV) than those receiving JL, indicating superior immunogenicity of RecombiMumps-G(dV). This effect was more pronounced when testing the sera for potency against RecombiMumps-G(dV) (FIG. 7B) where neutralizing antibody tiers in animals vaccinated with RecombiMumps-G(dV) were over 600-fold higher as compared to animals vaccinated with JL.

Since the intent of vaccination is not to protect individuals against the vaccine virus, but to protect them against circulating wild type viruses, sera from the 10 monkeys were tested against a wild type genotype G virus (Iowa-G) isolated from a mumps case during an outbreak at a university in Iowa. The data are shown in FIG. 7C.

Significantly greater titers following RecombiMumps-G(dV) vaccination were observed at all timepoints. During peak antibody production on day 45, anti-Iowa-G neutralizing antibody titers were nearly 10-fold higher in monkeys vaccinated with RecombiMumps-G(dV) as compared to monkeys vaccinated with JL and this effect persisted through day 60 where there was a greater than 4-fold difference.

These data indicate that RecombiMumps-G(dV) affords superior protection against contemporary circulating wild type mumps virus infection as compared to the JL vaccine that is currently being used globally.

In view of the many possible embodiments to which the principles of the disclosed subject matter may be applied, it should be recognized that the illustrated embodiments are only preferred examples of the disclosure and should not be taken as limiting the scope of the disclosure. Rather, the scope of the disclosure is defined by the following claims.

1. An isolated nucleic acid molecule comprising a cDNA sequence encoding a mumps virus nucleoprotein (N) gene, a mumps virus phosphoprotein (P) gene, a mumps virus matrix protein (M) gene, a mumps virus fusion protein (F) gene, a mumps virus small hydrophobic protein (SH) gene, a mumps virus hemagglutinin-neuraminidase protein (HN) gene and a mumps virus large protein (L) gene, wherein the N, P, M, SH and L genes comprise Jeryl Lynn strain mumps virus gene sequences, and the F and HN genes comprise genotype G mumps virus gene sequences.

2. The isolated nucleic acid molecule of claim 1, wherein the F gene is a synthetic gene comprising a consensus genotype G mumps virus F gene sequence, or the HN gene is a synthetic gene comprising a consensus genotype G mumps virus HN gene sequence, or both.

3. The isolated nucleic acid molecule of claim 1, wherein the P gene comprises at least one mutation that prevents expression of the mumps virus V protein.

4. The isolated nucleic acid molecule of claim 3, wherein the at least one mutation introduces one or more stop codons in the V protein open reading frame (ORF).

5. The isolated nucleic acid molecule of claim 3, wherein the at least one mutation does not prevent expression of the mumps virus I protein or the P protein.

6. The isolated nucleic acid molecule of claim 1, wherein the cDNA sequence encodes in the 5′ to 3′ direction the L gene, the HN gene, the SH gene, the F gene, the M gene, the P gene and the N gene.

7. The isolated nucleic acid molecule of claim 1, wherein:
the sequence of the N gene comprises nucleotides 146-1795 of SEQ ID NO: 1;
the sequence of the P gene comprises nucleotides 1979-3152 of SEQ ID NO: 1;
the sequence of the M gene comprises nucleotides 3264-4380 of SEQ ID NO: 1;
the sequence of the SH gene comprises nucleotides 6268-6441 of SEQ ID NO: 1;
the sequence of the L gene comprises nucleotides 8438-15223 of SEQ ID NO: 1;
the sequence of the F gene comprises SEQ ID NO: 2; and/or
the sequence of the HN gene comprises SEQ ID NO: 3.

8-13. (canceled)

14. The isolated nucleic acid molecule of claim 1, comprising the nucleotide sequence of SEQ ID NO: 1.

15. A plasmid comprising the isolated nucleic acid molecule of claim 1.

16. An isolated host cell comprising the plasmid of claim 15.

17. A recombinant mumps virus, wherein the genome of the recombinant mumps virus is encoded by the nucleic acid molecule of claim 1.

18. A recombinant mumps virus, wherein the genome of the recombinant mumps virus comprises a nucleoprotein (N) gene, a phosphoprotein (P) gene, a matrix protein (M) gene, a fusion protein (F) gene, a small hydrophobic protein (SH) gene, a hemagglutinin-neuraminidase protein (HN) gene and a large protein (L) gene, wherein the N, P, M, SH and L genes comprise Jeryl Lynn strain mumps virus gene sequences, and the F and HN genes comprise genotype G mumps virus gene sequences.

19. The recombinant mumps virus of claim 18, wherein the F gene is a synthetic gene comprising a consensus genotype G mumps virus F gene sequence, or the HN gene is a synthetic gene comprises a consensus genotype G mumps virus HN gene sequence, or both.

20. The recombinant mumps virus of claim 18, wherein the P gene comprises at least one mutation that prevents expression of the mumps virus V protein.

21. The recombinant mumps virus of claim 20, wherein the at least one mutation introduces one or more stop codons in the V protein open reading frame (ORF).

22. The recombinant mumps virus of claim 20, wherein the at least one mutation does not prevent expression of the mumps virus I protein or the P protein.

23. The recombinant mumps virus of claim 18, wherein the genome of the recombinant mumps virus comprises in the 5′ to 3′ direction the L gene, the HN gene, the SH gene, the F gene, the M gene, the P gene and the N gene.

24. The recombinant mumps virus of claim 18, wherein:
the N gene is encoded by nucleotides 146-1795 of SEQ ID NO: 1;
the P gene is encoded by nucleotides 1979-3152 of SEQ ID NO: 1;
the M gene is encoded by nucleotides 3264-4380 of SEQ ID NO: 1;
the SH gene is encoded by nucleotides 6268-6441 of SEQ ID NO: 1;
the L gene is encoded by nucleotides 8438-15223 of SEQ ID NO: 1;
the F gene is encoded by SEQ ID NO: 2; and/or
the HN gene is encoded by SEQ ID NO: 3.

25-30. (canceled)

31. The recombinant mumps virus of claim 18, wherein the genome of the virus is encoded by the nucleotide sequence of SEQ ID NO: 1.

32. The recombinant mumps virus of claim 18, wherein the virus is attenuated.

33. A composition comprising the recombinant mumps virus of claim 18 and a pharmaceutically acceptable carrier.

34. A method of eliciting an immune response against mumps virus in a subject, comprising administering to the subject an effective amount of the composition of claim 33.

35. The method of claim 34, wherein the composition is administered in a single dose.

36. The method of claim 34, wherein the composition is administered in multiple doses.

37. A collection of plasmids, comprising:
the plasmid of claim 15;
a plasmid comprising a mumps virus N gene ORF;
a plasmid comprising a mumps virus P gene ORF; and
a plasmid comprising a mumps virus L gene ORF.

38. The collection of claim 37, wherein:
the plasmid comprising the mumps virus N gene ORF comprises the nucleotide sequence of SEQ ID NO: 7;
the plasmid comprising the mumps virus P gene ORF comprises the nucleotides sequence of SEQ ID NO: 8; and/or
the plasmid comprising the mumps virus L gene ORF comprises the nucleotides sequence of SEQ ID NO: 9.

39-40. (canceled)

41. The collection of claim 37, wherein the plasmids comprise a T7 polymerase promoter.

42. A method of producing a recombinant mumps virus, comprising:
transfecting cultured cells with the collection of plasmids of claim 37;
incubating the transfected cells for a sufficient time to allow for mumps virus replication; and
collecting the recombinant mumps virus from the cell culture supernatant.

43. The method of claim 42, wherein the cultured cells express T7 polymerase.


SEQUENCE LISTING (9 sequences)

SEQ ID NO: 1 - DNA, 15384 bp, Artificial Sequence
  Synthetic nucleic acid construct
  gene (146)..(1795): Jeryl Lynn N gene
  misc_feature (1832)..(1835): mutations to introduce a restriction site
  gene (1979)..(3152): Jeryl Lynn V, P and I genes
  misc_feature (2459)..(2461): Stop codon
  misc_feature (2460)..(2460): mutation to introduce a stop codon
  misc_feature (2462)..(2462): mutation to introduce a stop codon
  misc_feature (2495)..(2495): mutation to introduce a stop codon
  misc_feature (2495)..(2497): stop codon
  misc_feature (2516)..(): mutation to introduce a stop codon
  misc_feature (2516)..(2518): stop codon
  gene (3264)..(4380): Jeryl Lynn M gene
  misc_feature (4463)..(4463): mutation to introduce a restriction site
  misc_feature (4466)..(4466): mutation to introduce a restriction site
  misc_feature (4469)..(4469): mutation to introduce a restriction site
  gene (4486)..(6162): Genotype G F gene
  gene (6268)..(6441): Jeryl Lynn SH gene
  misc_feature (6466)..(6466): mutation to introduce a restriction site
  misc_feature (6472)..(6473): mutations to introduce a restriction site
  gene (6614)..(8362): Genotype G HN gene
  misc_feature (8397)..(8397): mutation to introduce a restriction site
  misc_feature (8399)..(8399): mutation to introduce a restriction site
  gene (8438)..(15223): Jeryl Lynn L gene
  misc_feature (9634)..(9634): mutation to introduce a restriction site
accaaggggagaatgaatatgggatattggtagaacaaatagtgtaagaaacagtaagcccggaagtggtgttttgcgatttcgaggccgagctcgatcctcaccttccatcgtcgctagggggcattttgacactacctggaaaatgtcatctgtgctcaaggcatttgagcggttcacgatagaacaggaacttcaagacaggggtgaggagggttcaattccaccggagactttaaagtcagcagtcaaagtcttcgttattaacacacccaatcccaccacacgctatcagatgctaaacttttgcttaagaataatctgcagtcaaaatgctagggcatctcacagggtaggtgcattgataacattattctcacttccctcagcaggcatgcaaaatcatattagattagcagatagatcacccgaagctcagatagaacgctgtgagattgatggttttgagcctggtacatataggctgattccaaatgcacgcgccaatcttactgccaatgaaattgctgcctatgctttgcttgcagatgacctccctccaaccataaataatggaactccttacgtacatgcagatgttgaaggacagccatgtgatgagattgagcagttcctggatcggtgttacagtgtactaatccaggcttgggtaatggtctgtaaatgtatgacagcgtacgaccaacctgccgggtctgctgatcggcgatttgcgaaataccagcagcaaggtcgccttgaggcaagatacatgctgcaaccggaggcccaaaggttgattcaaactgccatcaggaaaagtcttgttgttagacagtaccttaccttcgaactccagttggcgagacggcagggattgctatcaaacagatactatgcaatggtgggtgacatcggaaagtacattgagaattcaggccttactgccttctttctcactctcaaatatgcactagggaccaaatggagtcctctatcattggctgcattcaccggtgaactcaccaagctccgatccttgatgatgttatatcgaggtctcggagaacaagccagataccttgctctgttagaggctccccaaataatggactttgcacccgggggctacccattgatattcagttatgctatgggagtcggtacagtcctagatgttcaaatgcgaaattacacttatgcacgacctttcctaaacggttattatttccagattggggttgagaccgcacgaagacaacaaggcactgttgacaacagagtagcagatgatctgggcctgactcctgagcaaagaactgaggtcactcagcttgttgacaggcttgcaaggggaagaggtgctgggataccaggtgggcctgtgaatccttttgttcctccggttcaacagcaacaacctgctgccgtatatgaggacattcctgcattggaggaatcagatgacgatggtgatgaagatggaggcgcaggattccaaaatggagtacaattaccagctgtaagacagggaggtcaaactgactttagagcacagcctttgcaagatccaattcaagcacaacttttcatgccattatatcctcaagtcagcaacatgccaaataatcagaatcatcagatcaatcgcatcggggggctggaacaccaagatttattacgatacaacgagaatggtgattcccaacaagatgcaaggggcgaacacgtaaacactttcccaaacaatcccaatcaaaacgcacagttgcaagtgggagactgggatgagtaaatcactgacatgatcaaactaaccccaatcgcaacacctgcaggacaatccagccacagctaactgcccaaatccactacattccattcatatttagtctttaagaaaaaattaggcccggaaagaattaggtccacgatcacaggcacaatcatttttatcgtgtttctttccgggcaagccatggatcaatttataaaacaggatgagaccggtgatttaattgagacaggaatgaatgttgcgaatcatttcctatccaccccaattcagggaaccaattcgctgagcaaggcctcaatcctccctggtgttgcacctgtactcattggcaatccagagcaaaagaacattcagcaccctaccgcatcacatcagggatccaagacaaagggcagaggctcaggagtcaggtccatcatagtctcaccctccgaagcaggcaatggagggactcagattcctgagcccctttttgcacaaacaggacagggtggtatagtcaccacagtttaccaggatccaactatccaaccaacaggttcataccgaagtgtggaattggcgaagatcggaaaagagagaatgattaatcgatttgttgagaaacctagaacctcaacgccggtgacagaatttaagaggggggccgggagcggctgctaatggccagacaatccaagaggagggcatagacggtaatggagcctcagctgggtctaaggagaggtccgggtctttgagtggtgcaaccctatatgctcacctatcactgccgcagcaagattccactcctgcaaatgtgggaattgccccgcaaagtgcgatcagtgcgaacgagattatggacctccttagggggatggatgctcgcctgcaacatcttgaacaaaaggtggacaaggtgcttgcacagggcagcatggtgacccaaataaagaatgaattatcaacagtaaagacaacattagcaacaattgaagggatgatggcaacagtaaagatcatggatcctggaaatccgacaggggtcccagttgatgagcttagaagaagttttagtgatcacgtgacaattgttagtggaccaggagatgtgtcgttcagctccagtgaaaaacccacactgtatttggatgagctggcgaggcccgtctccaagcctcgtcctgcaaagcagacaaaatcccaaccagtaaaggatttagcaggacagaaagtgatgattaccaaaatgatcactgattgtgtggctaatcctcaaatgaagcaggcgttcgagcaacgattggcaaaggccagcaccgaggatgctctgaacgatatcaagagagacatcatacgaagcgccatatgaattcaccaggagcaccagacgcgtggaaaaatctatgaactgagagccacaatgattccctattaaataaaaaataagcacgaacacaagtcaaatccaaccatagcagaaatggcaggatcacagatcaaaattcctcttccaaagccccccgattcagactctcaaagactaaatgccttccctgtcatcatggctcaagaaggcaaaggacgactccttagacaaatcaggcttaggaaaatattatcaggggatccgtctgatcagcaaattacatttgtgaatacatatggattcatccgtgccactccagaaacatccgagttcatctctgaatcatcacaacaaaaggtaactcctgtagtgacagcgtgcatgctgtcctttggtgccggaccagtgctagaagatccacaacatatgctcaaggctcttgatcagacagacattagggttcggaaaacagcaagtgataaagagcagatcttattcgagatcaaccgcatccccaatctattcaggcattatcaaatatctgcggaccatctgattcaggccagctccgataaatatgtcaaatcaccagcaaaattgattgcaggagtaaattacatctactgtgttacattcttatctgtgacagtttgttctgcctcactcaagtttcgagttgcgcgcccattgcttgctgcacggtccagattagtaagagcagttcagatggaaattttgcttcgggtaacttgcaaaaaagattctcaaatggcaaagagcatgttaaatgaccctgatggagaagggtgcattgcatccgtgtggttccacctatgtaatctgtgcaaaggcagaaataaacttagaagttacgatgaaaattattttgcttctaagtgccgtaagatgaatctgacagtcagcataggagatatgtggggaccaaccattctagtccatgcaggcggtcacattccgacaactgcaaaaccttttttcaactcaagaggctgggtctgccacccaatccaccaatcatcaccatcgttggcgaagaccctatggtcatctgggtgtgaaatcaaggctgccagtgctattctccagggttcagactatgcatcacttgcaaagactgatgacataatatattcgaagataaaagtcgataaagacgcggccaactacaaaggagtatcctggagtccattcaggaagtctgcctcaatgagaaacctatgagaatttcctctatttccactgatgcctataggagaatcaacaatcaagcaaatttgaccggtggtaattcgtttaaacttatagaaaaaataagcctagaaggatatcctacttctcgactttccaactttgaaaatagaatagatcagtaatcatgaaggtttctttagttacttgcttgggctttgcagtcttttcattttccatatgtgtgaatatcaatatcttgcagcaaattggatatatcaagcaacaagtcaggcaactgagctattactcacaaagttcaagctcctacatagtggtcaagcttttaccgaatatccaacccactgataacagctgtgaattcaagagtgtaactcaatacaataagaccttgagtaatttgcttcttccaattgcagaaaacataaataatattgcatcgccctcacctggatcaagacgtcataaaaggtttgctggcattgccattggcattgctgcactcggtgttgcaaccgcagcacaagtaaccgccgctgtctcattagttcaagcacagacaaatgcacgcgcaatagcggcgatgaaaaattcaatacaggcaactaatcgagcagtcttcgaagtgaaagaaggcacccaacagttagctatagcggtacaagcaatacagaaccacatcaatactattatgaacacccaattgaacaatatgtcctgtcagattcttgataaccagcttgcaacctccctgggattatacctaacagaattaacaacagtgtttcagccacaattaattaatccggcattgtcaccgattagtatacaagccttgaggtctttgcttggaagtatgacacctgcagtggttcaagcaacattatctacttcaatttctgctgctgaaatactaagtgccggtctaatggagggtcagattgtttctgttctgctggatgagatgcagatgatagttaagataaatattccaaccattgtcacacaatcaaatgcattggtgattgacttctactcaatttcgagctttattaataatcaggaatccataattcaattaccagacaggatcttggagatcgggaatgaacaatggagctatccagcaaaaaattgtaagttgacaagacacaacatattctgccaatacaatgaggcagagaggctgagcttagaatcaaaactatgccttgcaggcaatataagtgcctgtgtgttctcacccatagcagggagttatatgaggcgatttgtagcactggatggaacaattgttgcaaactgtcgaagtctaacgtgtctatgcaagagtccatcttatcctatataccaacctgaccatcatgcagtcacgaccattgatctaaccgcatgtcagacgttgtccctagacggattggatttcagcattgtctctctaagcaacatcacttacgctgagaaccttaccatttcattgtctcagacaatcaatactcaacccattgacatatcaactgaactgatcaaggtcaatgcatccctccaaaatgccgttaagtacataaaggagagcaaccatcaactccaatctgtgagtataaattctaaaatcggagctataatcatagcagccttagttttgagcatcctgtcaatgatcatttcactgttgttttgctgctgggcttacattgcaactaaagagatcagaagaatcaacttcaaaacaaatcatatcaacacaatatcaagtagtgtcgatgatctcatcaggtactaatcttagattggtgattcgtcctgcaattttaaaagatttagaaaaaaactaaaataagaatgaatctcctagggtcgtaacgtcacgtcaccctgccgtcgcactatgccggcaatccaacctcccttatacctaacatttctagtgctaatccttctctatctcatcataaccctgtatgtctggactatattgactattaactataagacggcggtgcgatatgcagcactgtaccagcgatccttctctcgctggggttttgatcactcactctagaaagatccccaattaggacaagtcgcgatcgctcacgctagaacaagctgcattcaaatgaagctgtgctaccatgagacataaagaaaaaagcaagccagaacaaacctaggatcataacacaatacagaatattagctgctatcacaactgtgttccggccactaagaaaatggagccctcgaaattcttcacaatatcggacagtgccacctttgcacctgggcctgttagcaatgcggctaacaagaagacattccgaacctgcttccgaatactggcactatctgtacaagctgtcacccttatattagttattgtcactttaggtgagcttgtaaggatgatcaatgatcaaggcttgagcaatcagttgtcttcaattacagacaagataagagagtcagctactatgattgcatctgctgtgggagtaatgaatcaagttattcatggagtaacggtatccttacccctacaaattgagggaaaccaaaatcaattgttagccacacttgccacaatctgcaccagccaaaaacaagtctcaaactgctctacaaacatccccttagtcaatgacctcaggtttataaatgggatcaataaattcatcattgaagattacgcaactcatgatttctctatcggccatccactcaatatgcccagctttatcccaactgcaacttcacccaatggttgcacaagaattccatccttttctttaggtaagacacactggtgctacacacataatgtaattaatgccaactgcaaggaccatacttcgtctaaccaatatgtgtccatggggattctcgttcagaccgcgtcaggttatcctatgttcaaaaccttaaaaatccaatatctcagtgatggcctgaatcggaaaagctgctcaattgcaacagtccctgatgggtgcgcgatgtactgttatgtctcaactcaacttgaaaccgacgactatgcggggtccagtccacccacccaaaaacttaccctgttattctataatgacaccgtcacagaaaggacaatatctccatctggtcttgaagggaattgggctactttggtgccaggagtggggagtgggatatattttgagaataagttgatcttccctgcatatggtggtgtcttgcccaatagtacactcggggttaaatcagcaagagaatttttccggcctgttaatccatataatccatgttcaggaccacaacaagatttagaccagcgtgctttgaggtcatacttcccaagttatttctctaatcgaagaatacagagtgcatttcttgtctgtgcctggaatcagatcctagttacaaattgtgagctagttgtcccctcaagcaatcagacaatgatgggtgcagaagggagagttttattgatcaataatcgactattatattatcagagaagtaccagctggtggccgtatgaactcctctacgagatatcattcacatttacaaactctggtccatcatctgtaaatatgtcctggatacctatatattcattcactcgtcctggttcaggcaattgcagtggtgaaaatgtgtgcccgactgcttgtgtgtcaggggtttatcttgatccctggccattaactccatatagccaccaatcaggtattaacagaaatttctatttcacaggtgctctattaaattcaagtacaactagagtaaatcctaccctttatgtctctgctcttaataatcttaaagtattagccccatatggtactcaaggactgtttgcctcgtacaccacaaccacctgctttcaagataccggtgatgctagtgtgtattgtgtttatattatggaactagcatcaaatattgttggagaattccaaattctacctgtgctaactagattgactatcacttgagttgtagtgaatgtagcaggaagctttacgggcgcgcctcatttcttattgattattaagaaaaaacaggccagaatggcgggcctaaatgagatactcctacccgaagtacatttaaactcccccatcgttagatataagcttttctactatatattgcatggccagttaccaaatgacttggagccggatgacttgggcccattagcaaatcagaattggaaggcaattcgagctgaagaatcacaggttcatgcacgtttaaaacagatcagagtagaactcattgcaaggattcctagtctccggtggacccgatctcaaagagagattgccatactcatttggccaagaatacttccaatactgcaagcatatgatcttcggcaaagtatgcaattgcccacagtgtgggagaaactgactcaatccacggttaatcttataagtgacggtctagaacgggttgtattacacatcagcaatcaactaacaggcaagcctaacttgtttaccagatctcgagccggacaagacacaaaagattactcaattccatccactagagagctatctcaaatatggttcaacaatgagtggagtgggtctgtaaagacctggcttatgattaaatatagaatgaggcagctaatcacaaatcaaaagacaggtgagttaacagatctagtaaccattgtggatactaggtccactctatgcattattactccagaattagtcgctttatactccagtgagcacaaagcattaacgtacctcacctttgaaatggtattaatggtcactgatatgttagagggacggctgaatgtttcttctctgtgcacagctagtcattatctgtcccctttaaaaaagagaatcgaagttctcctgacattagttgatgaccttgcactactcatgggggataaagtatacggtattgtctcttcacttgagagttttgtttacgcccaattacagtatggtgatcctgttatagacattaaaggtacattctatggatttatatgtaatgagattctcgacctactgactgaagacaacatctttactgaagaagaggctaataaggttcttctggacttaacatcacaatttgacaatctatcccctgatttaactgctgagctcctctgcattatgagactttggggccatcccaccttaactgccagccaagcagcatccaaggtccgagagtccatgtgcgctcctaaggtattagactttcaaacaataatgaaaaccctggctttctttcacgcaatcctaattaacggttataggaggagccataatggaatctggccgcctaccactcttcatggcaatgcccccaaaagcctcattgagatgcggcatgataattcagagcttaagtatgagtatgtcctcaagaattggaaaagtatatctatgttaagaatacacaaatgctttgatgcatcacctgatgaagatctcagcatattcatgaaggataaggcaataagctgtccaaggcaagactggatgggagtatttaggaggagcctgattaaacagcgctatcgtgacgcgaatcggcctctaccacaaccatttaaccggagactgctgttgaattttctagaggatgaccgattcgatcctattaaagagcttgagtatgtcaccagtggagaatatcttagggaccctgaattttgtgcatcttactctctcaaggagaaggagataaaggctacaggtcgtatatttgcaaaaatgacaaagagaatgagatcgtgccaagtaattgcagaatcattgttagccaatcacgcaggtaaattaatgagagagaatggggttgtcttagaccagttaaaattaacaaaatctttattaactatgaaccaaattggtattatatcagagcacagccgaagatccaccgctgacaacatgactttagcacactccggttcaaataagcacaggattaataatagtcaattcaagaagaataaagacaataaacatgagatgcctgatgatgggtttgagatagcagcctgcttcctaacaactgacctcacaaaatactgcttgaattggaggtaccaggtcatcatcccctttgcgcgtacattgaattcaatgtatggtataccccacttgtttgaatggatacatttaaggctgatgcgaagcactctttatgtcggtgatcccttcaatcctccatcagatcctacccaacttgaccttgatacagccctcaatgatgatatatttatagtttcccctcgtggcggaatcgagggtttatgtcaaaaattatggactatgatttccatctcaacaatcatattgtccgcaactgaggcaaacactagagtaatgagcatggttcagggcgataaccaagcaattgcaatcaccactagagtagtacgttcgctcagtcattccgagaagaaggagcaagcctataaagcaagtaaattattctttgaaaggcttagagctaacaaccatggaattggacaccacttaaaagaacaagaaacaatccttagttctgatttcttcatttacagtaagagggtgttttacaaaggtcgaatcttgactcaagcgttaaagaacgtgagcaagatgtgcttaacagctgatatactgggggattgttcacaagcatcatgctccaatttagctaccactgtaatgcgcctgactgagaatggggtcgagaaagatttgtgttatttcctaaatgcattcatgacaattagacaattatgttatgatctagtatttccccaaactaaatctcttagtcaggacattactaatgcttatcttaatcatccaatacttatctcaagattgtgtctattaccatctcaattggggggcttaaactttctttcatgtagtcgcctgtttaatagaaacataggagatccactagtgtctgcaattgctgatgtgaaacgattaattaaagcgggctgtctagatatctgggtcctgtacaacatccttggaaggaggccaggaaaaggtaagtggagcactctggcagctgatccctatactttaaacatagattatttagtcccttcaacaacttttttgaagaaacatgcccaatatacattgatggaacggagtgttaatcccatgctccgcggagtatttagtgaaaatgcagcagaggaggaagaagaactcgcacagtatctattagatcgcgaagtagtcatgcccagggttgcacatgttatacttgctcagtctagttgcggtagaagaaaacagatccaaggttacttggattctactagaactattattaggtattcactggaggtaaggccactgtcagcaaagaagctgaatacagtaatagaatataacttattgtacctgtcctacaatttggagattattgaaaaacccaatatagtccaaccttttttgaatgcaatcaatgttgatacttgtagcatcgatatagctaggtcccttagaaaattatcctgggcaactttacttaatggacgtcccatcgagggattagaaacacctgatcctattgaattggtacatgggtgtttaataatcgggtcagatgagtgtgagcattgcagtagtggtgatgacaaattcacctggtttttcctccctaaggggataaggttagatgatgatccggcatctaacccacccatcagagtaccttatatcggatccaaaacagatgaacgaagggttgcatcaatggcttatatcaaaggggcatcagtatcacttaaatcagcactcagattagcgggggtatatatatgggctttcggagatacagaggaatcatggcaggatgcctatgagttagcttccactcgtgttaatctcacactagagcaattgcaatctctcactcctttaccaacatctgccaacttagtccacagattggatgatggcactactcaattaaaatttaccccagcaagctcctatgcattctctagctttgttcatatatctaacgactgtcaaattcttgagatcgatgatcaggtaacggattctaacctgatttaccagcaagtcatgattactggccttgctctaattgagacatggaataatcctccaatcaacttctccgtttatgaaaccacattacacttgcacacaggctcatcttgctgtataagacctgtcgagtcttgtgtagtaaatccgcctttacttcctgtccctctcattaatgttcctcaaatgaataaatttgtatatgatcctgaaccacttagtttgttagaaatggaaaaaattgaggatattgcttatcaaaccagaattggtggtttagatcaaatcccgcttctggaaaaaatacccttactagctcaccttaccgccaagcagatggtaaatagcatcactgggcttgatgaagcaacatctataatgaatgatgctgtagttcaagcagactatactagcaattggattagtgaatgctgctatacttacattgactctgtgtttgtttactccggctgggcattattattggaactttcataccaaatgtattacctaagaattcaaggcatacaaggaatcctagactatgtgtatatgaccttgaggaggataccaggaatggccataacaggcatctcatccacaattagtcaccctcgtatactcagaagatgcatcaatttggatgtcatagccccaatcaattctccacacatagcttcactggattacacaaaattgagcatagatgcagtaatgtggggaaccaagcaggtgttgaccaacatttcgcaaggtatcgattatgagatagttgttccttctgaaagccaacttacactcagtgatagagtcctaaatctagttgctcgaaaattatcactactggcaatcatctgggccaattacaactatcctccgaaggttaaaggtatgtcacctgaagacaaatgtcaggctttaactacacatctactccaaactgttgaatatgtcgagtacattcagattgaaaagacaaacatcaggaggatgattattgagccaaaattaactgcctaccctagtaatttgttttacctctctcgaaagctgcttaatgctattcgagactcagaagaaggacaattcctgattgcatcctattataacagttttggatatctggaaccgatattaatggaatctaaaatattcaatctgagttcatccgaatcagcatctcttacagaatttgatttcatcctcaacttggaattgtccgacgccagccttgagaaatactctctcccaagtttgcttatgacggctgagaatatggataacccatttcctcaacccccacttcatcacgttctcagaccactaggtttgtcatccacctcatggtataaaacaatcagtgttttaaattatattagccatatgaagatatctgacggtgcccatctatacttggcagagggaagtggagcctctatgtcacttatagaaactttcttgcccggggaaacaatatggtacaacagcctgttcaatagtggtgagaatccccctcaacgtaatttcgcccctttgcccacccagtttattgaaagtgtcccctatagattgattcaggcaggtatagcagcaggaaatggcatagtgcaaagtttctatccgctctggaacggaaacagcgatataactgacttaagcacgaaaactagtgttgaatacattatccacaaggtaggagctgatacttgtgcattagttcatgtggatttggaaggtgtacctggctcaatgaacagcatgttggagagagctcaagtacatgcgctgctaattacagtgactgtattaaaaccaggcggcttactaatcttgaaagcttcatgggaaccttttaatcgattttcctttttactcacagtactctggcaattcttttccacaattaggatcttgcgatcttcatactccgatccgaataatcacgaggtttacataatagccacattggcagttgatcccaccacatcctcctttacaactgctctgaatagggcacgcaccctgaatgaacagggcttttcactcatcccacctgaattagtgagtgagtactggaggaagcgtgttgaacaaggacagattatacaggactgtatagataaagttatatcagagtgtgtcagagatcaatatctggcagacaacaacattatcctccaagcgggaggtactccgagcacaagaaaatggttggatcttcctgactattcttcgttcaatgaattacaatctgaaatggccagactcataacaattcatcttaaagaggtaatagaaatcctaaagggccaagcatcagatcatgacaccctattatttacttcatacaacgtaggtcccctcggaaaaataaatacaatactcagattgattgttgagagaattcttatgtatactgtgaggaactggtgtatcttgcctacccaaactcgtctcaccttacgacaatctatcgagcttggagagtttagactaagagatgtgataacacccatggagattctaaaactatcccccaacaggaaatatctgaagtctgcattaaatcaatcaacattcaatcatctaatgggagaaacatctgacatattgttaaaccgagcttatcagaagagaatttggaaagctattgggtgtgtaatctattgctttggtttgctcaccccagatgttgaaggttctgagcgcattgatgttgataatgacatacctgattatgatattcacggggacataatttaaatcgactaaagactcctctggcattacacatcaccaaaaagtgccgaactaacatccaaattcttctaaaccgcacacgacctcgaacaatcataaccacatcagtattaaatctaggagatccttttaagaaaaaattgattttactttctccccttggt

SEQ ID NO: 2 - DNA, 1617 bp, Artificial Sequence
  Synthetic nucleic acid
atgaaggtttctttagttacttgcttgggctttgcagtcttttcattttccatatgtgtgaatatcaatatcttgcagcaaattggatatatcaagcaacaagtcaggcaactgagctattactcacaaagttcaagctcctacatagtggtcaagcttttaccgaatatccaacccactgataacagctgtgaattcaagagtgtaactcaatacaataagaccttgagtaatttgcttcttccaattgcagaaaacataaataatattgcatcgccctcacctggatcaagacgtcataaaaggtttgctggcattgccattggcattgctgcactcggtgttgcaaccgcagcacaagtaaccgccgctgtctcattagttcaagcacagacaaatgcacgcgcaatagcggcgatgaaaaattcaatacaggcaactaatcgagcagtcttcgaagtgaaagaaggcacccaacagttagctatagcggtacaagcaatacagaaccacatcaatactattatgaacacccaattgaacaatatgtcctgtcagattcttgataaccagcttgcaacctccctgggattatacctaacagaattaacaacagtgtttcagccacaattaattaatccggcattgtcaccgattagtatacaagccttgaggtctttgcttggaagtatgacacctgcagtggttcaagcaacattatctacttcaatttctgctgctgaaatactaagtgccggtctaatggagggtcagattgtttctgttctgctggatgagatgcagatgatagttaagataaatattccaaccattgtcacacaatcaaatgcattggtgattgacttctactcaatttcgagctttattaataatcaggaatccataattcaattaccagacaggatcttggagatcgggaatgaacaatggagctatccagcaaaaaattgtaagttgacaagacacaacatattctgccaatacaatgaggcagagaggctgagcttagaatcaaaactatgccttgcaggcaatataagtgcctgtgtgttctcacccatagcagggagttatatgaggcgatttgtagcactggatggaacaattgttgcaaactgtcgaagtctaacgtgtctatgcaagagtccatcttatcctatataccaacctgaccatcatgcagtcacgaccattgatctaaccgcatgtcagacgttgtccctagacggattggatttcagcattgtctctctaagcaacatcacttacgctgagaaccttaccatttcattgtctcagacaatcaatactcaacccattgacatatcaactgaactgatcaaggtcaatgcatccctccaaaatgccgttaagtacataaaggagagcaaccatcaactccaatctgtgagtataaattctaaaatcggagctataatcatagcagccttagttttgagcatcctgtcaatgatcatttcactgttgttttgctgctgggcttacattgcaactaaagagatcagaagaatcaacttcaaaacaaatcatatcaacacaatatcaagtagtgtcgatgatctcatcaggtactaa

SEQ ID NO: 3 - DNA, 1749 bp, Artificial Sequence
  Synthetic nucleic acid
atggagccctcgaaattcttcacaatatcggacagtgccacctttgcacctgggcctgttagcaatgcggctaacaagaagacattccgaacctgcttccgaatactggcactatctgtacaagctgtcacccttatattagttattgtcactttaggtgagcttgtaaggatgatcaatgatcaaggcttgagcaatcagttgtcttcaattacagacaagataagagagtcagctactatgattgcatctgctgtgggagtaatgaatcaagttattcatggagtaacggtatccttacccctacaaattgagggaaaccaaaatcaattgttagccacacttgccacaatctgcaccagccaaaaacaagtctcaaactgctctacaaacatccccttagtcaatgacctcaggtttataaatgggatcaataaattcatcattgaagattacgcaactcatgatttctctatcggccatccactcaatatgcccagctttatcccaactgcaacttcacccaatggttgcacaagaattccatccttttctttaggtaagacacactggtgctacacacataatgtaattaatgccaactgcaaggaccatacttcgtctaaccaatatgtgtccatggggattctcgttcagaccgcgtcaggttatcctatgttcaaaaccttaaaaatccaatatctcagtgatggcctgaatcggaaaagctgctcaattgcaacagtccctgatgggtgcgcgatgtactgttatgtctcaactcaacttgaaaccgacgactatgcggggtccagtccacccacccaaaaacttaccctgttattctataatgacaccgtcacagaaaggacaatatctccatctggtcttgaagggaattgggctactttggtgccaggagtggggagtgggatatattttgagaataagttgatcttccctgcatatggtggtgtcttgcccaatagtacactcggggttaaatcagcaagagaatttttccggcctgttaatccatataatccatgttcaggaccacaacaagatttagaccagcgtgctttgaggtcatacttcccaagttatttctctaatcgaagaatacagagtgcatttcttgtctgtgcctggaatcagatcctagttacaaattgtgagctagttgtcccctcaagcaatcagacaatgatgggtgcagaagggagagttttattgatcaataatcgactattatattatcagagaagtaccagctggtggccgtatgaactcctctacgagatatcattcacatttacaaactctggtccatcatctgtaaatatgtcctggatacctatatattcattcactcgtcctggttcaggcaattgcagtggtgaaaatgtgtgcccgactgcttgtgtgtcaggggtttatcttgatccctggccattaactccatatagccaccaatcaggtattaacagaaatttctatttcacaggtgctctattaaattcaagtacaactagagtaaatcctaccctttatgtctctgctcttaataatcttaaagtattagccccatatggtactcaaggactgtttgcctcgtacaccacaaccacctgctttcaagataccggtgatgctagtgtgtattgtgtttatattatggaactagcatcaaatattgttggagaattccaaattctacctgtgctaactagattgactatcacttga

SEQ ID NO: 4 - DNA, 2051 bp, Artificial Sequence
  Synthetic nucleic acid
  misc_feature (1)..(205): Jeryl Lynn sequence
  misc_feature (22)..(29): PmeI site
  gene (106)..(1722): Genotype G F gene ORF
  misc_feature (1723)..(2051): Jeryl Lynn sequence
  misc_feature (2026)..(2033): AsiSI site
  misc_feature (2041)..(2046): MluI site
  misc_feature (2045)..(2050): SalI site
gggcgcgcctgcaggtaattcgtttaaacttatagaaaaaataagcctagaaggatatcctacttctcgactttccaactttgaaaatagaatagatcagtaatcatgaaggtttctttagttacttgcttgggctttgcagtcttttcattttccatatgtgtgaatatcaatatcttgcagcaaattggatatatcaagcaacaagtcaggcaactgagctattactcacaaagttcaagctcctacatagtggtcaagcttttaccgaatatccaacccactgataacagctgtgaattcaagagtgtaactcaatacaataagaccttgagtaatttgcttcttccaattgcagaaaacataaataatattgcatcgccctcacctggatcaagacgtcataaaaggtttgctggcattgccattggcattgctgcactcggtgttgcaaccgcagcacaagtaaccgccgctgtctcattagttcaagcacagacaaatgcacgcgcaatagcggcgatgaaaaattcaatacaggcaactaatcgagcagtcttcgaagtgaaagaaggcacccaacagttagctatagcggtacaagcaatacagaaccacatcaatactattatgaacacccaattgaacaatatgtcctgtcagattcttgataaccagcttgcaacctccctgggattatacctaacagaattaacaacagtgtttcagccacaattaattaatccggcattgtcaccgattagtatacaagccttgaggtctttgcttggaagtatgacacctgcagtggttcaagcaacattatctacttcaatttctgctgctgaaatactaagtgccggtctaatggagggtcagattgtttctgttctgctggatgagatgcagatgatagttaagataaatattccaaccattgtcacacaatcaaatgcattggtgattgacttctactcaatttcgagctttattaataatcaggaatccataattcaattaccagacaggatcttggagatcgggaatgaacaatggagctatccagcaaaaaattgtaagttgacaagacacaacatattctgccaatacaatgaggcagagaggctgagcttagaatcaaaactatgccttgcaggcaatataagtgcctgtgtgttctcacccatagcagggagttatatgaggcgatttgtagcactggatggaacaattgttgcaaactgtcgaagtctaacgtgtctatgcaagagtccatcttatcctatataccaacctgaccatcatgcagtcacgaccattgatctaaccgcatgtcagacgttgtccctagacggattggatttcagcattgtctctctaagcaacatcacttacgctgagaaccttaccatttcattgtctcagacaatcaatactcaacccattgacatatcaactgaactgatcaaggtcaatgcatccctccaaaatgccgttaagtacataaaggagagcaaccatcaactccaatctgtgagtataaattctaaaatcggagctataatcatagcagccttagttttgagcatcctgtcaatgatcatttcactgttgttttgctgctgggcttacattgcaactaaagagatcagaagaatcaacttcaaaacaaatcatatcaacacaatatcaagtagtgtcgatgatctcatcaggtactaatcttagattggtgattcgtcctgcaattttaaaagatttagaaaaaaactaaaataagaatgaatctcctagggtcgtaacgtcacgtcaccctgccgtcgcactatgccggcaatccaacctcccttatacctaacatttctagtgctaatccttctctatctcatcataaccctgtatgtctggactatattgactattaactataagacggcggtgcgatatgcagcactgtaccagcgatccttctctcgctggggttttgatcactcactctagaaagatccccaattaggacaagtcgcgatcgctcacgctacgcgtcgacg

SEQ ID NO: 5 - DNA, 1967 bp, Artificial Sequence
  Synthetic nucleic acid
  misc_feature (1)..(162): Jeryl Lynn sequence
  misc_feature (1)..(7): NotI site
  gene (163)..(1911): Genotype G HN gene ORF
  misc_feature (1912)..(1967): Jerly Lynn sequence
  misc_feature (1942)..(1949): AscI site
  misc_feature (1957)..(1962): KpnI site
  misc_feature (1962)..(1967): XhoI site
gcggccgccaagtcgcgatcgctcacgctagaacaagctgcattcaaatgaagctgtgctaccatgagacataaagaaaaaagcaagccagaacaaacctaggatcataacacaatacagaatattagctgctatcacaactgtgttccggccactaagaaaatggagccctcgaaattcttcacaatatcggacagtgccacctttgcacctgggcctgttagcaatgcggctaacaagaagacattccgaacctgcttccgaatactggcactatctgtacaagctgtcacccttatattagttattgtcactttaggtgagcttgtaaggatgatcaatgatcaaggcttgagcaatcagttgtcttcaattacagacaagataagagagtcagctactatgattgcatctgctgtgggagtaatgaatcaagttattcatggagtaacggtatccttacccctacaaattgagggaaaccaaaatcaattgttagccacacttgccacaatctgcaccagccaaaaacaagtctcaaactgctctacaaacatccccttagtcaatgacctcaggtttataaatgggatcaataaattcatcattgaagattacgcaactcatgatttctctatcggccatccactcaatatgcccagctttatcccaactgcaacttcacccaatggttgcacaagaattccatccttttctttaggtaagacacactggtgctacacacataatgtaattaatgccaactgcaaggaccatacttcgtctaaccaatatgtgtccatggggattctcgttcagaccgcgtcaggttatcctatgttcaaaaccttaaaaatccaatatctcagtgatggcctgaatcggaaaagctgctcaattgcaacagtccctgatgggtgcgcgatgtactgttatgtctcaactcaacttgaaaccgacgactatgcggggtccagtccacccacccaaaaacttaccctgttattctataatgacaccgtcacagaaaggacaatatctccatctggtcttgaagggaattgggctactttggtgccaggagtggggagtgggatatattttgagaataagttgatcttccctgcatatggtggtgtcttgcccaatagtacactcggggttaaatcagcaagagaatttttccggcctgttaatccatataatccatgttcaggaccacaacaagatttagaccagcgtgctttgaggtcatacttcccaagttatttctctaatcgaagaatacagagtgcatttcttgtctgtgcctggaatcagatcctagttacaaattgtgagctagttgtcccctcaagcaatcagacaatgatgggtgcagaagggagagttttattgatcaataatcgactattatattatcagagaagtaccagctggtggccgtatgaactcctctacgagatatcattcacatttacaaactctggtccatcatctgtaaatatgtcctggatacctatatattcattcactcgtcctggttcaggcaattgcagtggtgaaaatgtgtgcccgactgcttgtgtgtcaggggtttatcttgatccctggccattaactccatatagccaccaatcaggtattaacagaaatttctatttcacaggtgctctattaaattcaagtacaactagagtaaatcctaccctttatgtctctgctcttaataatcttaaagtattagccccatatggtactcaaggactgtttgcctcgtacaccacaaccacctgctttcaagataccggtgatgctagtgtgtattgtgtttatattatggaactagcatcaaatattgttggagaattccaaattctacctgtgctaactagattgactatcacttgagttgtagtgaatgtagcaggaagctttacgggcgcgcctcatttcggtacctcgag

SEQ ID NO: 6 - DNA, 443 bp, Artificial Sequence
  Synthetic nucleic acid
  misc_feature (1)..(6): native ClaI site
  misc_feature (72)..(74): non-native stop codon
  misc_feature (108)..(110): non-native stop codon
  misc_feature (129)..(131): non-native stop codon
  misc_feature (437)..(443): native EcoO109I site
atcgatttgttgagaaacctagaacctcaacgccggtgacagaatttaagaggggggccgggagcggctgctaatggccagacaatccaagaggagggcatagacggtaatggagcctcagctgggtctaaggagaggtccgggtctttgagtggtgcaaccctatatgctcacctatcactgccgcagcaagattccactcctgcaaatgtgggaattgccccgcaaagtgcgatcagtgcgaacgagattatggacctccttagggggatggatgctcgcctgcaacatcttgaacaaaaggtggacaaggtgcttgcacagggcagcatggtgacccaaataaagaatgaattatcaacagtaaagacaacattagcaacaattgaagggatgatggcaacagtaaagatcatggatcctggaaatccgacaggggtccc

SEQ ID NO: 7 - DNA, 1737 bp, Artificial Sequence
  Synthetic nucleic acid
  misc_feature (1)..(27): overhang sequence
  misc_feature (19)..(24): NcoI site
  gene (28)..(1677): Jeryl Lynn N gene ORF
  misc_feature (1678)..(1737): overhang sequence
  misc_feature (1714)..(1719): XhoI site
ggtcgacgcgttgacactccatggaaaatgtcatctgtgctcaaggcatttgagcggttcacgatagaacaggaacttcaagacaggggtgaggagggttcaattccaccggagactttaaagtcagcagtcaaagtcttcgttattaacacacccaatcccaccacacgctatcagatgctaaacttttgcttaagaataatctgcagtcaaaatgctagggcatctcacagggtaggtgcattgataacattattctcacttccctcagcaggcatgcaaaatcatattagattagcagatagatcacccgaagctcagatagaacgctgtgagattgatggttttgagcctggtacatataggctgattccaaatgcacgcgccaatcttactgccaatgaaattgctgcctatgctttgcttgcagatgacctccctccaaccataaataatggaactccttacgtacatgcagatgttgaaggacagccatgtgatgagattgagcagttcctggatcggtgttacagtgtactaatccaggcttgggtaatggtctgtaaatgtatgacagcgtacgaccaacctgccgggtctgctgatcggcgatttgcgaaataccagcagcaaggtcgccttgaggcaagatacatgctgcaaccggaggcccaaaggttgattcaaactgccatcaggaaaagtcttgttgttagacagtaccttaccttcgaactccagttggcgagacggcagggattgctatcaaacagatactatgcaatggtgggtgacatcggaaagtacattgagaattcaggccttactgccttctttctcactctcaaatatgcactagggaccaaatggagtcctctatcattggctgcattcaccggtgaactcaccaagctccgatccttgatgatgttatatcgaggtctcggagaacaagccagataccttgctctgttagaggctccccaaataatggactttgcacccgggggctacccattgatattcagttatgctatgggagtcggtacagtcctagatgttcaaatgcgaaattacacttatgcacgacctttcctaaacggttattatttccagattggggttgagaccgcacgaagacaacaaggcactgttgacaacagagtagcagatgatctgggcctgactcctgagcaaagaactgaggtcactcagcttgttgacaggcttgcaaggggaagaggtgctgggataccaggtgggcctgtgaatccttttgttcctccggttcaacagcaacaacctgctgccgtatatgaggacattcctgcattggaggaatcagatgacgatggtgatgaagatggaggcgcaggattccaaaatggagtacaattaccagctgtaagacagggaggtcaaactgactttagagcacagcctttgcaagatccaattcaagcacaacttttcatgccattatatcctcaagtcagcaacatgccaaataatcagaatcatcagatcaatcgcatcggggggctggaacaccaagatttattacgatacaacgagaatggtgattcccaacaagatgcaaggggcgaacacgtaaacactttcccaaacaatcccaatcaaaacgcacagttgcaagtgggagactgggatgagtaaatcactgacatgatcaaactaaccccaatcgcaacactcgagggacaatacgcgtcgacg

SEQ ID NO: 8 - DNA, 1281 bp, Artificial Sequence
  Synthetic nucleic acid
  misc_feature (1)..(20): overhang sequence
  misc_feature (19)..(24): NcoI site
  gene (21)..(1196): Jeryl Lynn P gene ORF
  misc_feature (481)..(482): insertion
  misc_feature (1197)..(1281): overhang sequence
  misc_feature (1258)..(1263): XhoI site
ggtcgacgcgtgggcaagccatggatcaatttataaaacaggatgagaccggtgatttaattgagacaggaatgaatgttgcgaatcatttcctatccaccccaattcagggaaccaattcgctgagcaaggcctcaatcctccctggtgttgcacctgtactcattggcaatccagagcaaaagaacattcagcaccctaccgcatcacatcagggatccaagacaaagggcagaggctcaggagtcaggtccatcatagtctcaccctccgaagcaggcaatggagggactcagattcctgagcccctttttgcacaaacaggacagggtggtatagtcaccacagtttaccaggatccaactatccaaccaacaggttcataccgaagtgtggaattggcgaagatcggaaaagagagaatgattaatcgatttgttgagaaacctagaacctcaacgccggtgacagaatttaagaggggggggccgggagcggctgctcaaggccagacaatccaagaggagggcatagacgggaatggagcctcagctgggtccaaggagaggtccgggtctttgagtggtgcaaccctatatgctcacctatcactgccgcagcaagattccactcctgcaaatgtgggaattgccccgcaaagtgcgatcagtgcgaacgagattatggacctccttagggggatggatgctcgcctgcaacatcttgaacaaaaggtggacaaggtgcttgcacagggcagcatggtgacccaaataaagaatgaattatcaacagtaaagacaacattagcaacaattgaagggatgatggcaacagtaaagatcatggatcctggaaatccgacaggggtcccagttgatgagcttagaagaagttttagtgatcacgtgacaattgttagtggaccaggagatgtgtcgttcagctccagtgaaaaacccacactgtatttggatgagctggcgaggcccgtctccaagcctcgtcctgcaaagcagacaaaatcccaaccagtaaaggatttagcaggacagaaagtgatgattaccaaaatgatcactgattgtgtggctaatcctcaaatgaagcaggcgttcgagcaacgattggcaaaggccagcaccgaggatgctctgaacgatatcaagagagacatcatacgaagcgccatatgaattcaccaggagcaccagactcaaggaaaaatctatgaactgagagccacaatgattccctctcgagtagagcgacgcgtcgacg

SEQ ID NO: 9 - DNA, 6899 bp, Artificial Sequence
  Synthetic nucleic acid
  misc_feature (1)..(45): upstream sequence
  misc_feature (1)..(8): AscI site
  gene (46)..(6831): Jeryl Lynn L gene ORF
  misc_feature (3527)..(3532): AatII site
  misc_feature (6581)..(6586): NcoI site
  misc_feature (6832)..(6899): downstream sequence
  misc_feature (6893)..(6899): PstI site
ggcgcgcctcatttcttattgattattaagaaaaaacaggccagaatggcgggcctaaatgagatactcctacccgaagtacatttaaactcccccatcgttagatataagcttttctactatatattgcatggccagttaccaaatgacttggagccggatgacttgggcccattagcaaatcagaattggaaggcaattcgagctgaagaatcacaggttcatgcacgtttaaaacagatcagagtagaactcattgcaaggattcctagtctccggtggacccgatctcaaagagagattgccatactcatttggccaagaatacttccaatactgcaagcatatgatcttcggcaaagtatgcaattgcccacagtgtgggagaaactgactcaatccacggttaatcttataagtgacggtctagaacgggttgtattacacatcagcaatcaactaacaggcaagcctaacttgtttaccagatctcgagccggacaagacacaaaagattactcaattccatccactagagagctatctcaaatatggttcaacaatgagtggagtgggtctgtaaagacctggcttatgattaaatatagaatgaggcagctaatcacaaatcaaaagacaggtgagttaacagatctagtaaccattgtggatactaggtccactctatgcattattactccagaattagtcgctttatactccagtgagcacaaagcattaacgtacctcacctttgaaatggtattaatggtcactgatatgttagagggacggctgaatgtttcttctctgtgcacagctagtcattatctgtcccctttaaaaaagagaatcgaagttctcctgacattagttgatgaccttgcactactcatgggggataaagtatacggtattgtctcttcacttgagagttttgtttacgcccaattacagtatggtgatcctgttatagacattaaaggtacattctatggatttatatgtaatgagattctcgacctactgactgaagacaacatctttactgaagaagaggctaataaggttcttctggacttaacatcacaatttgacaatctatcccctgatttaactgctgagctcctctgcattatgagactttggggccatcccaccttaactgccagccaagcagcatccaaggtccgagagtccatgtgcgctcctaaggtattagactttcaaacaataatgaaaaccctggctttctttcacgcaatcctaattaacggttataggaggagccataatggaatctggccgcctaccactcttcatggcaatgcccccaaaagcctcattgagatgcggcatgataattcagagcttaagtatgagtatgtcctcaagaattggaaaagtatatctatgttaagaatacacaaatgctttgatgcatcacctgatgaagatctcagcatattcatgaaggataaggcaataagctgtccaaggcaagactggatgggagtatttaggaggagcctgattaaacagcgctatcgtgacgcgaatcggcctctaccacaaccatttaaccggagactgctgttgaattttctagaggatgaccgattcgatcctattaaagagcttgagtatgtcaccagtggagaatatcttagggaccctgaattttgtgcatcttactctctcaaggagaaggagataaaggctacaggtcgtatatttgcaaaaatgacaaagagaatgagatcgtgccaagtaattgcagaatcattgttagccaatcacgcaggtaaattaatgagagagaatggggttgtcttagaccagttaaaattaacaaaatctttattaactatgaaccaaattggtattatatcagagcacagccgaagatccaccgctgacaacatgactttagcacactccggttcaaataagcacaggattaataatagtcaattcaagaagaataaagacaataaacatgagatgcctgatgatgggtttgagatagcagcctgcttcctaacaactgacctcacaaaatactgcttgaattggaggtaccaggtcatcatcccctttgcgcgtacattgaattcaatgtatggtataccccacttgtttgaatggatacatttaaggctgatgcgaagcactctttatgtcggtgatcccttcaatcctccatcagatcctacccaacttgaccttgatacagccctcaatgatgatatatttatagtttcccctcgtggcggaatcgagggtttatgtcaaaaattatggactatgatttccatctcaacaatcatattgtccgcaactgaggcaaacactagagtaatgagcatggttcagggcgataaccaagcaattgcaatcaccactagagtagtacgttcgctcagtcattccgagaagaaggagcaagcctataaagcaagtaaattattctttgaaaggcttagagctaacaaccatggaattggacaccacttaaaagaacaagaaacaatccttagttctgatttcttcatttacagtaagagggtgttttacaaaggtcgaatcttgactcaagcgttaaagaacgtgagcaagatgtgcttaacagctgatatactgggggattgttcacaagcatcatgctccaatttagctaccactgtaatgcgcctgactgagaatggggtcgagaaagatttgtgttatttcctaaatgcattcatgacaattagacaattatgttatgatctagtatttccccaaactaaatctcttagtcaggacattactaatgcttatcttaatcatccaatacttatctcaagattgtgtctattaccatctcaattggggggcttaaactttctttcatgtagtcgcctgtttaatagaaacataggagatccactagtgtctgcaattgctgatgtgaaacgattaattaaagcgggctgtctagatatctgggtcctgtacaacatccttggaaggaggccaggaaaaggtaagtggagcactctggcagctgatccctatactttaaacatagattatttagtcccttcaacaacttttttgaagaaacatgcccaatatacattgatggaacggagtgttaatcccatgctccgcggagtatttagtgaaaatgcagcagaggaggaagaagaactcgcacagtatctattagatcgcgaagtagtcatgcccagggttgcacatgttatacttgctcagtctagttgcggtagaagaaaacagatccaaggttacttggattctactagaactattattaggtattcactggaggtaaggccactgtcagcaaagaagctgaatacagtaatagaatataacttattgtacctgtcctacaatttggagattattgaaaaacccaatatagtccaaccttttttgaatgcaatcaatgttgatacttgtagcatcgatatagctaggtcccttagaaaattatcctgggcaactttacttaatggacgtcccatcgagggattagaaacacctgatcctattgaattggtacatgggtgtttaataatcgggtcagatgagtgtgagcattgcagtagtggtgatgacaaattcacctggtttttcctccctaaggggataaggttagatgatgatccggcatctaacccacccatcagagtaccttatatcggatccaaaacagatgaacgaagggttgcatcaatggcttatatcaaaggggcatcagtatcacttaaatcagcactcagattagcgggggtatatatatgggctttcggagatacagaggaatcatggcaggatgcctatgagttagcttccactcgtgttaatctcacactagagcaattgcaatctctcactcctttaccaacatctgccaacttagtccacagattggatgatggcactactcaattaaaatttaccccagcaagctcctatgcattctctagctttgttcatatatctaacgactgtcaaattcttgagatcgatgatcaggtaacggattctaacctgatttaccagcaagtcatgattactggccttgctctaattgagacatggaataatcctccaatcaacttctccgtttatgaaaccacattacacttgcacacaggctcatcttgctgtataagacctgtcgagtcttgtgtagtaaatccgcctttacttcctgtccctctcattaatgttcctcaaatgaataaatttgtatatgatcctgaaccacttagtttgttagaaatggaaaaaattgaggatattgcttatcaaaccagaattggtggtttagatcaaatcccgcttctggaaaaaatacccttactagctcaccttaccgccaagcagatggtaaatagcatcactgggcttgatgaagcaacatctataatgaatgatgctgtagttcaagcagactatactagcaattggattagtgaatgctgctatacttacattgactctgtgtttgtttactccggctgggcattattattggaactttcataccaaatgtattacctaagaattcaaggcatacaaggaatcctagactatgtgtatatgaccttgaggaggataccaggaatggccataacaggcatctcatccacaattagtcaccctcgtatactcagaagatgcatcaatttggatgtcatagccccaatcaattctccacacatagcttcactggattacacaaaattgagcatagatgcagtaatgtggggaaccaagcaggtgttgaccaacatttcgcaaggtatcgattatgagatagttgttccttctgaaagccaacttacactcagtgatagagtcctaaatctagttgctcgaaaattatcactactggcaatcatctgggccaattacaactatcctccgaaggttaaaggtatgtcacctgaagacaaatgtcaggctttaactacacatctactccaaactgttgaatatgtcgagtacattcagattgaaaagacaaacatcaggaggatgattattgagccaaaattaactgcctaccctagtaatttgttttacctctctcgaaagctgcttaatgctattcgagactcagaagaaggacaattcctgattgcatcctattataacagttttggatatctggaaccgatattaatggaatctaaaatattcaatctgagttcatccgaatcagcatctcttacagaatttgatttcatcctcaacttggaattgtccgacgccagccttgagaaatactctctcccaagtttgcttatgacggctgagaatatggataacccatttcctcaacccccacttcatcacgttctcagaccactaggtttgtcatccacctcatggtataaaacaatcagtgttttaaattatattagccatatgaagatatctgacggtgcccatctatacttggcagagggaagtggagcctctatgtcacttatagaaactttcttgcccggggaaacaatatggtacaacagcctgttcaatagtggtgagaatccccctcaacgtaatttcgcccctttgcccacccagtttattgaaagtgtcccctatagattgattcaggcaggtatagcagcaggaaatggcatagtgcaaagtttctatccgctctggaacggaaacagcgatataactgacttaagcacgaaaactagtgttgaatacattatccacaaggtaggagctgatacttgtgcattagttcatgtggatttggaaggtgtacctggctcaatgaacagcatgttggagagagctcaagtacatgcgctgctaattacagtgactgtattaaaaccaggcggcttactaatcttgaaagcttcatgggaaccttttaatcgattttcctttttactcacagtactctggcaattcttttccacaattaggatcttgcgatcttcatactccgatccgaataatcacgaggtttacataatagccacattggcagttgatcccaccacatcctcctttacaactgctctgaatagggcacgcaccctgaatgaacagggcttttcactcatcccacctgaattagtgagtgagtactggaggaagcgtgttgaacaaggacagattatacaggactgtatagataaagttatatcagagtgtgtcagagatcaatatctggcagacaacaacattatcctccaagcgggaggtactccgagcacaagaaaatggttggatcttcctgactattcttcgttcaatgaattacaatctgaaatggccagactcataacaattcatcttaaagaggtaatagaaatcctaaagggccaagcatcagatcatgacaccctattatttacttcatacaacgtaggtcccctcggaaaaataaatacaatactcagattgattgttgagagaattcttatgtatactgtgaggaactggtgtatcttgcctacccaaactcgtctcaccttacgacaatctatcgagcttggagagtttagactaagagatgtgataacacccatggagattctaaaactatcccccaacaggaaatatctgaagtctgcattaaatcaatcaacattcaatcatctaatgggagaaacatctgacatattgttaaaccgagcttatcagaagagaatttggaaagctattgggtgtgtaatctattgctttggtttgctcaccccagatgttgaaggttctgagcgcattgatgttgataatgacatacctgattatgatattcacggggacataatttaaatcgactaaagactcctctggcattacacatcaccaaaaagtgccgaactaacatccaaattctgcag